SEQUENCE LISTING (44 sequences)

SEQ ID NO: 1 - protein, 12 aa, Homo sapiens
GDSISSTSYYWG

SEQ ID NO: 2 - protein, 16 aa, Homo sapiens
SFYYSGSTYYNPSLKS

SEQ ID NO: 3 - protein, 10 aa, Homo sapiens
MQLWSYDVDV

SEQ ID NO: 4 - protein, 14 aa, Homo sapiens
TGTSSDVGGYDYVA

SEQ ID NO: 5 - protein, 7 aa, Homo sapiens
DVSERPS

SEQ ID NO: 6 - protein, 11 aa, Homo sapiens
SSYTSTTTLVV

SEQ ID NO: 7 - protein, 120 aa, Homo sapiens
QLQLQESGPGLVKPSETLSLTCTVSGDSISSTSYYWGWIRQPPGKGLEWIGSFYYSGSTYYNPSLKSRVTISVDTSKNQFSLKLNSVTAADTAVYYCARMQLWSYDVDVWGQGTTVTVSS

SEQ ID NO: 8 - protein, 111 aa, Homo sapiens
QSALTQPASVSGSPGQSITISCTGTSSDVGGYDYVAWYQQHPGKVPKLMIYDVSERPSGVSNRFSGSKSGNTASLTISGLQAEDEADYYCSSYTSTTTLVVFGGGTKLSVL

SEQ ID NO: 9 - DNA, 360 bp, Homo sapiens
cagctgcagctgcaggagtcgggcccaggactggtgaagccttcggagaccctgtccctcacctgcactgtctctggtgactccatcagcagtactagttactactggggctggatccgccagcccccagggaaggggctggagtggattgggagtttctattatagtgggagcacctactacaacccgtccctcaagagtcgagtcaccatttccgtagacacgtccaagaaccagttctccctgaagctgaactctgtgaccgccgcagacacggctgtgtattactgtgcgaggatgcagctatggtcgtacgatgtggacgtctggggccaagggaccacggtcaccgtctcctca

SEQ ID NO: 10 - DNA, 333 bp, Homo sapiens
cagtctgccctgactcaacctgcctccgtgtctgggtctcctggacagtcgatcaccatctcctgcactggaaccagcagtgacgttggtgggtatgactatgtcgcctggtaccaacaacacccaggcaaagtccccaaactcatgatttatgatgtcagtgagcggccctcaggggtttctaatcgcttctctggctccaagtctggcaacacggcctccctgaccatctctgggctccaggctgaggacgaggctgattattactgcagctcatatacaagcaccaccactctcgttgtgttcggcggagggaccaagctgtccgtcctg

SEQ ID NO: 11 - protein, 10 aa, Homo sapiens
GFTFSSHAMS

SEQ ID NO: 12 - protein, 17 aa, Homo sapiens
TITGGGGSIYYADSVKG

SEQ ID NO: 13 - protein, 10 aa, Homo sapiens
NRAGEGYFDY

SEQ ID NO: 14 - protein, 11 aa, Homo sapiens
GGDNIGNKDVH

SEQ ID NO: 15 - protein, 7 aa, Homo sapiens
RDSNRPS

SEQ ID NO: 16 - protein, 8 aa, Homo sapiens
QVWDSIWV

SEQ ID NO: 17 - protein, 119 aa, Homo sapiens
EVQLLESGGGLVQPGGSLRLSCAASGFTFSSHAMSWVRQAPGKGLEWVSTITGGGGSIYYADSVKGRFTISRDNSKNTLYLQMNSLRAEDTAVYYCAKNRAGEGYFDYWGQGTLVTVSS

SEQ ID NO: 18 - protein, 105 aa, Homo sapiens
SYELTQPLSVSVALGQTARITCGGDNIGNKDVHWYQQKPGQAPVLVIYRDSNRPSGIPEGFSGSNSGNTATLTISRAQAGDEADYYCQVWDSIWVFGGGTKLTVL

SEQ ID NO: 19 - DNA, 357 bp, Homo sapiens
gaggtgcagctgttggagtctgggggaggcttggtacagcctggggggtccctgagactgtcctgcgcagcctctggattcacctttagcagccatgccatgagctgggtccgccaggctccagggaaggggctggagtgggtctcaactattactggtggtggtggtagcatatactacgcagactccgtgaagggccggttcaccatctccagagacaattccaagaacacgctgtatctgcaaatgaacagcctgagagccgaggacacggccgtatattattgtgcgaaaaaccgcgctggggagggttactttgactactggggccagggaaccctggtcaccgtctcctca

SEQ ID NO: 20 - DNA, 315 bp, Homo sapiens
tcctatgagctgactcagccactctcagtgtcagtggccctgggacagacggccaggattacctgtgggggagacaacattggaaataaagatgtgcactggtaccagcagaagccaggccaggcccctgtgctggtcatctatagggatagcaaccggccctctgggatccctgagggattctctggctccaactcggggaacacggccaccctgaccatcagcagagcccaagccggggatgaggctgactattactgtcaggtgtgggacagcatttgggtgttcggcggagggaccaagctgaccgtccta

SEQ ID NO: 21 - protein, 10 aa, Rattus norvegicus
GFTFTTYYIS

SEQ ID NO: 22 - protein, 17 aa, Rattus norvegicus
YINMGSGGTNYNEKFKG

SEQ ID NO: 23 - protein, 6 aa, Rattus norvegicus
IGYFDY

SEQ ID NO: 24 - protein, 16 aa, Rattus norvegicus
RSSQSLLDSDGGTYLY

SEQ ID NO: 25 - protein, 7 aa, Rattus norvegicus
LVSTLGS

SEQ ID NO: 26 - protein, 9 aa, Rattus norvegicus
MQLTHWPYT

SEQ ID NO: 27 - protein, 115 aa, Artificial Sequence
  synthetic
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 28 - protein, 112 aa, Artificial Sequence
  Synthetic
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIK

SEQ ID NO: 29 - DNA, 345 bp, Artificial Sequence
  Synthetic
caggtgcagctggtccagtctggagctgaggtgaagaaacccggcagctccgtgaaggtcagttgcaaagcatcaggcttcacttttaccacatactatatctcttgggtgaggcaggcacctggacagggcctggagtacctgggctatattaacatggggtccggcgggaccaactacaatgaaaagttcaaagggcgggtgactatcaccgcagacaagtccacatctactgcctatatggagctgtctagtctgagatccgaagacacagccgtctactattgtgctattatcggctactttgattattgggggcagggaacgatggtgacagtctcaagc

SEQ ID NO: 30 - DNA, 336 bp, Artificial Sequence
  Synthetic
gacgtggtcatgactcagtctcccctgtccctgcctgtgaccctgggacagccagcctctatcagttgccgaagctcccagtcactgctggacagcgatgggggtacatacctgtattggtttcagcagagaccaggacagagcccccggcggctgatctacctggtgtccaccctgggatctggagtccctgacaggttctcaggaagcggctccgggaccgacttcaccctgaagattagccgcgtggaggccgaagatgtgggggtctactattgtatgcagctgactcactggccatatacctttggacagggcacaaagctggagatcaag

SEQ ID NO: 31 - protein, 446 aa, Homo sapiens
EVQLLESGGGLVQPGGSLRLSCAASGFTFSSHAMSWVRQAPGKGLEWVSTITGGGGSIYYADSVKGRFTISRDNSKNTLYLQMNSLRAEDTAVYYCAKNRAGEGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 467 aa, Artificial Sequence
  Synthetic
SYELTQPLSVSVALGQTARITCGGDNIGNKDVHWYQQKPGQAPVLVIYRDSNRPSGIPEGFSGSNSGNTATLTISRAQAGDEADYYCQVWDSIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECSGGGGSGGGGSQLQLQESGPGLVKPSETLSLTCTVSGDSISSTSYYWGWIRQPPGKGLEWIGSFYYSGSTYYNPSLKSRVTISVDTSKNQFSLKLNSVTAADTAVYYCARMQLWSYDVDVWGQGTTVTVSSGGGGSGGGGSGGGGSQSALTQPASVSGSPGQSITISCTGTSSDVGGYDYVAWYQQHPGKVPKLMIYDVSERPSGVSNRFSGSKSGNTASLTISGLQAEDEADYYCSSYTSTTTLVVFGGGTKLSVL

SEQ ID NO: 33 - protein, 711 aa, Artificial Sequence
  Synthetic
EVQLLESGGGLVQPGGSLRLSCAASGFTFSSHAMSWVRQAPGKGLEWVSTITGGGGSIYYADSVKGRFTISRDNSKNTLYLQMNSLRAEDTAVYYCAKNRAGEGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSQLQLQESGPGLVKPSETLSLTCTVSGDSISSTSYYWGWIRQPPGKGLEWIGSFYYSGSTYYNPSLKSRVTISVDTSKNQFSLKLNSVTAADTAVYYCARMQLWSYDVDVWGQGTTVTVSSGGGGSGGGGSGGGGSQSALTQPASVSGSPGQSITISCTGTSSDVGGYDYVAWYQQHPGKVPKLMIYDVSERPSGVSNRFSGSKSGNTASLTISGLQAEDEADYYCSSYTSTTTLVVFGGGTKLSVL

SEQ ID NO: 34 - protein, 211 aa, Homo sapiens
SYELTQPLSVSVALGQTARITCGGDNIGNKDVHWYQQKPGQAPVLVIYRDSNRPSGIPEGFSGSNSGNTATLTISRAQAGDEADYYCQVWDSIWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 35 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 37 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 246 aa, Artificial Sequence
  Synthetic
QLQLQESGPGLVKPSETLSLTCTVSGDSISSTSYYWGWIRQPPGKGLEWIGSFYYSGSTYYNPSLKSRVTISVDTSKNQFSLKLNSVTAADTAVYYCARMQLWSYDVDVWGQGTTVTVSSGGGGSGGGGSGGGGSQSALTQPASVSGSPGQSITISCTGTSSDVGGYDYVAWYQQHPGKVPKLMIYDVSERPSGVSNRFSGSKSGNTASLTISGLQAEDEADYYCSSYTSTTTLVVFGGGTKLSVL

SEQ ID NO: 39 - protein, 5 aa, Artificial Sequence
  Synthetic
GGGGS

SEQ ID NO: 40 - protein, 10 aa, Artificial Sequence
  Synthetic
GGGGSGGGGS

SEQ ID NO: 41 - protein, 15 aa, Artificial Sequence
  Synthetic
GGGGSGGGGSGGGGS

SEQ ID NO: 42 - protein, 20 aa, Artificial Sequence
  Synthetic
GGGGSGGGGSGGGGSGGGGS

SEQ ID NO: 43 - protein, 5 aa, Artificial Sequence
  Synthetic
TGGGG

SEQ ID NO: 44 - protein, 5 aa, Artificial Sequence
  Synthetic
SGGGG

(19) United States
(12) Patent Application Publication
(10) Pub. No.: US 2021/0115138 A1
(43) Pub. Date: Apr. 22, 2021

(54) NOVEL BISPECIFIC PD-1/LAG-3 ANTIBODY MOLECULES

(71) Applicant: WuXi Biologics Ireland Limited, Dublin (IE)

(72) Inventors: Zhuozhi Wang, Shanghai (CN); Yong Zheng, Shanghai (CN); Jing Li, Shanghai (CN); Qiong Wu, Shanghai (CN)

(73) Assignee: WuXi Biologics Ireland Limited, Dublin (IE)

(21) Appl. No.: 16/981,894

(22) PCT Filed: Mar. 19, 2019
(86) PCT No.: PCT/CN2019/078665

§ 371 Date: Sep. 17, 2020

(30) Foreign Application Priority Data
Mar. 20, 2018 (CN) PCT/CN2018/079691

(51) Int. Cl. C07K 16/28; A61P 35/00

(52) U.S. Cl. CPC C07K 16/2803; C07K 16/2818; A61P 35/00; C07K 2317/31; C07K 2317/55; A61K 2039/505; C07K 2317/92; C07K 2317/21; C07K 2317/33; C07K 2317/76; C07K 2317/622

(57) ABSTRACT

Anti-LAG-3/PD-1 bispecific antibody molecules, isolated polynucleotides encoding the same, pharmaceutical compositions comprising the same, and the uses thereof are provided.

### PRIORITY CLAIM

The present application claims priority to PCT Application Number PCT/CN2018/079691, filed on Mar. 20, 2018.

### FIELD OF THE INVENTION

The present disclosure generally relates to novel bispecific antibody molecules directed to human PD-1 and human LAG-3.

### BACKGROUND

Bispecific antibodies are growing to be the new category of therapeutic antibodies. They can bind two different targets or two different epitopes on a target, creating additive or synergistic effect superior to the effect of individual antibodies. A lot of antibody engineering efforts have been put into designing new bispecific formats, such as DVD-Ig, CrossMab, BiTE etc. (Spiess et al. Molecular Immunology, 67(2), pp. 95-106 (2015).). However, these formats may potentially have various limitations in stability, solubility, short half-life, and immunogenicity.

Increasing evidences from preclinical and clinical results have shown that targeting immune checkpoints is becoming the most promising approach to treat patients with cancers. Programmed cell death 1 (PD-1), one of immune-checkpoint proteins, play a major role in limiting the activity of T cells that provide a major immune resistance mechanism by which tumor cells escaped immune surveillance. The interaction of PD-1 expressed on activated T cells, and PD-L1 expressed on tumor cells negatively regulate immune response and damp anti-tumor immunity.

Lymphocyte-activation gene 3 (CD223), also known as LAG-3, is a type I transmembrane protein that is a member of the immune-globulin superfamily (IgSF).

LAG-3 is a cell surface molecule expressed on activated T cells, NK cells, B cells and plasmacytoid dendritic cells, but not on resting T cells. LAG-3 shares approximately 20% amino acid sequence homology with CD4, but binds to MHC class II with higher affinity, providing negative regulation of T cell receptor signaling.

Blockade of LAG-3 in vitro augments T cell proliferation and cytokine production, and LAG-3-deficient mice have a defect in the downregulation of T cell responses induced by the superantigen staphylococcal enterotoxin B, by peptides or by Sendai virus infection. LAG-3 is expressed on both activated natural Treg (nTreg) and induced CD4⁺FoxP3⁺ Treg (iTreg) cells, where expression levels are higher than that observed on activated effector CD4⁺ T cells. Blockade of LAG-3 on Treg cells abrogates Treg cell suppressor function whereas ectopic expression of LAG-3 in non-Treg CD4⁺ T cells confers suppressive activity. On the basis of the immunomodulatory role of LAG-3 on T cell function in chronic infection and cancer, the predicted mechanism of action for LAG-3-specific monoclonal antibodies is to inhibit the negative regulation of tumor-specific effector T cells.

In 2017, there were only three potential antagonist antibodies that regulate LAG-3 function and anti-tumor immune responses in early clinical developments for the treatment of advanced solid tumors. These antibodies are described in patent applications US 20110150892 A1, US 20170101472 A1 and WO 2015138920 A1, and referred hereinafter as BMK1, BMK7 and BMK5 respectively. BMK8, as described herein, is humanized version of chimeric antibody BMK5. BMK1, BMK7 and BMK8 serve as benchmark antibodies in the context of the application. Accordingly, there remains a need for anti-human LAG-3 antibodies with improved efficacy, such as high binding affinity, low cross-family reactions and good stability. In this application, the inventors have generated a series of antibodies and fully human antibodies against LAG-3 utilizing humanized rats. The antibodies of the instant application have high binding affinity, specifically binding to human LAG-3 protein without cross-family reactions, and are potent to modulate immune responses.

Despite of the development of therapeutics targeting the targets respectively, there is a significant need for novel bispecific therapeutics that can act on both targets.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides novel bispecific PD-1/LAG-3 antibody molecules, amino acid and nucleotide sequences thereof, and uses thereof.

In one aspect, the present disclosure provides herein a bispecific antibody molecule comprising a LAG-3-binding domain and a PD-1-binding domain, wherein:
the LAG-3-binding domain comprises:
   1, 2, or 3 heavy chain complementarity determining region (CDR) sequences selected from the group consisting of: SEQ ID NOs: 1-3; and/or
   1, 2, or 3 light chain CDR sequences selected from the group consisting of: SEQ ID NOs: 4-6, and
the PD-1-binding domain comprises:
   1, 2, or 3 heavy chain complementarity determining region (CDR) sequences selected from the group consisting of: SEQ ID NOs: 11-13; and/or
   1, 2, or 3 light chain CDR sequences selected from the group consisting of: SEQ ID NOs: 14-16,
the LAG-3-binding domain comprises one independently selected from the group consisting of: a Fab and a single chain Fv antibody (scFv); and
the PD-1-binding domain comprises one independently selected from the group consisting of: a Fab and a scFv.

In certain embodiments, the LAG-3-binding domain comprises a Fab.

In certain embodiments, the PD-1-binding domain comprises a Fab.

In certain embodiments, the LAG-3-binding domain comprises a scFv.

In certain embodiments, the PD-1-binding domain comprises a scFv.

In certain embodiments, the LAG-3-binding domain comprises a heavy chain variable region comprising 1, 2, or 3 CDR sequences selected from SEQ ID NOs: 1-3, and/or a light chain variable region comprising 1, 2, or 3 CDR sequences selected from SEQ ID NOs: 4-6.

In certain embodiments, the LAG-3-binding domain comprises a heavy chain variable region comprising SEQ ID NO: 7, and a homologous sequence thereof having at least 80% sequence identity yet retaining specific binding affinity to LAG-3.

In certain embodiments, the LAG-3-binding domain comprises a light chain variable region comprising SEQ ID NO: 8, and a homologous sequence thereof having at least 80% sequence identity yet retaining specific binding affinity to LAG-3.

In certain embodiments, the LAG-3-binding domain comprises a heavy chain variable region comprising SEQ ID NO: 7 and a light chain variable region comprising SEQ ID NO: 8.

In certain embodiments, the PD-1-binding domain comprises a heavy chain variable region comprising 1, 2, or 3 CDR sequences selected from SEQ ID NOs: 11-13, and/or a light chain variable region comprising 1, 2, or 3 CDR sequences selected from SEQ ID NOs: 14-16.

In certain embodiments, the PD-1-binding domain comprises a heavy chain variable region of SEQ ID NO: 17 or a homologous sequence thereof having at least 80% sequence identity yet retaining specific binding affinity to PD-1.

In certain embodiments, the PD-1-binding domain comprises a light chain variable region of SEQ ID NO: 18, or a homologous sequence thereof having at least 80% sequence identity yet retaining specific binding affinity to PD-1.

In certain embodiments, the PD-1-binding domain comprises a heavy chain variable region comprising SEQ ID NO: 17 and a light chain variable region comprising SEQ ID NO: 18.

In certain embodiments, the LAG-3-binding domain further comprises one or more amino acid residue substitutions or modifications yet retains specific binding affinity to LAG-3, and/or the PD-1-binding domain further comprises one or more amino acid residue substitutions or modifications yet retains specific binding affinity to PD-1.

In certain embodiments, at least one of the substitutions or modifications is in one or more of the CDR sequences, and/or in one or more of the VH or VL sequences but not in any of the CDR sequences.

In certain embodiments, the bispecific antibody molecule further comprises an immunoglobulin (Ig) constant region, optionally a constant region of human Ig, or optionally a constant region of human IgG.

In certain embodiments, the LAG-3-binding domain is operably linked to N terminus or the C terminus of the PD-1-binding domain.

In certain embodiments, the LAG-3-binding domain comprises a scFv and the PD-1-binding domain comprises a Fab.

In certain embodiments, the LAG-3-binding scFv comprises the sequence of SEQ ID NO: 38, and the PD-1-binding Fab comprises a heavy chain variable region comprising the sequence of SEQ ID NO: 17 and a light chain variable region comprising the sequence of SEQ ID NO: 18.

In certain embodiments, the LAG-3-binding scFv is operably linked to the C terminus of the light chain constant region following the PD-1-binding Fab.

In certain embodiments, the bispecific antibody comprises a heavy chain in the format of: VH(anti-PD-1)-CH1-Hinge-CH2-CH3, which is associated with the light chain in the format of: VL(anti-PD-1)-CL-spacer-scFv(anti-LAG-3).

In certain embodiments, the bispecific antibody molecule comprising a heavy chain comprising the sequence of SEQ ID NO: 31 and a light chain comprising the sequence of SEQ ID NO: 32.

In certain embodiments, the LAG-3-binding scFv is operably linked to the C terminus of the heavy chain constant region following the PD-1-binding Fab.

In certain embodiments, the bispecific antibody comprises a heavy chain in the format of: VH(anti-PD-1)-CH1-Hinge-CH2-CH3-spacer-scFv(anti-LAG-3), which is associated with the light chain VL(anti-PD-1)-CL.

In certain embodiments, the bispecific antibody molecule comprising a heavy chain comprising the sequence of SEQ ID NO: 33 and a light chain comprising the sequence of SEQ ID NO: 34.

In certain embodiments, the LAG-3-binding domain and/or the PD-1-binding domain is fully human or humanized.

In certain embodiments, the bispecific antibody molecule as provided herein is linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In another aspect, the present disclosure provides a pharmaceutical composition comprising the bispecific antibody molecule as provided herein, and a pharmaceutically acceptable carrier.

In another aspect, the present disclosure provides an isolated polynucleotide encoding the bispecific antibody molecule as provided herein.

In certain embodiments, the isolated polynucleotide comprising a nucleotide sequence selecting from a group consisting of SEQ ID NO: 9, 10, 19, 20, 29 and 30, and/or a homologous sequence thereof having at least 80% (e.g. at least 85%, 88%, 90%, 92%, 93%, 94%, 95%, 96%, 97%, 98%, or 99%) sequence identity, and/or a variant thereof having only degenerate substitutions.

In another aspect, the present disclosure provides a vector comprising the isolated polynucleotide as provided herein.

In another aspect, the present disclosure provides a host cell comprising the vector as provided herein.

In another aspect, the present disclosure provides a method of expressing the bispecific antibody molecule as provided herein, comprising culturing the host cell as provided herein under the condition at which the vector as provided herein is expressed.

In another aspect, the present disclosure provides a method of treating a disease or condition in a subject that would benefit from upregulation of an immune response, comprising administering to the subject a therapeutically effective amount of the bispecific antibody molecule as provided herein or the pharmaceutical composition as provided herein.

In certain embodiments, the disease or condition that would benefit from upregulation of an immune response is selected from the group consisting of cancer, a viral infection, a bacterial infection, a protozoan infection, a helminth infection, asthma associated with impaired airway tolerance, a neurological disease, multiple sclerosis, and an immunosuppressive disease.

In certain embodiments, the disease or condition is PD-1-related and/or LAG-3-related.

In certain embodiments, the PD-1-related disease or condition is cancer or infectious disease.

In certain embodiments, the LAG-3-related disease or condition is cancer.

In certain embodiments, the cancer is lymphoma, lung cancer, liver cancer, cervical cancer, colon cancer, breast cancer, ovarian cancer, pancreatic cancer, melanoma, glioblastoma, prostate cancer, esophageal cancer or gastric cancer.

In certain embodiments, the subject is human.

In certain embodiments, the administration is via oral, nasal, intravenous, subcutaneous, sublingual, or intramuscular administration.

In another aspect, the present disclosure provides a method of modulating LAG-3 activity in a LAG-3-expressing cell, comprising exposing the LAG-3-expressing cell to the bispecific antibody molecule as provided herein.

In another aspect, the present disclosure provides use of the bispecific antibody molecule as provided herein in the manufacture of a medicament for treating a disease or condition that would benefit from upregulation of an immune response.

In another aspect, the present disclosure provides use of the bispecific antibody molecule as provided herein in the manufacture of a medicament for treating a disease or condition that is PD-1 and/or LAG-3-related.

### BRIEF DESCRIPTION OF FIGURES

FIG. 1 shows W365-G14 and W365-G15 bound to human PD-1 protein.

FIG. 2 shows W365-G14 and W365-G15 bound to human LAG-3 protein.

FIG. 3 shows W365-G14 and W365-G15 bound to cell surface human PD-1.

FIG. 4 shows W365-G14 and W365-G15 bound to cell surface human LAG-3.

FIG. 5 shows W365-G14 and W365-G15 bound to cell surface cynomolgus PD-1.

FIG. 6 shows W365-G14 and W365-G15 bound to cell surface cynomolgus LAG-3.

FIG. 7A shows W365-G14 and W365-G15 did not bind to mouse PD-1.

FIG. 7B shows W365-G14 and W365-G15 did not bind to mouse LAG-3.

FIG. 8A shows W365-G14 and W365-G15 did not bind to human CTLA-4 protein.

FIG. 8B shows W365-G14 and W365-G15 did not bind to human CD28 protein.

FIG. 8C shows W365-G14 and W365-G15 did not bind to human CD4 protein.

FIG. 9 shows W365-G14 and W365-G15 bound to human PD-1 and LAG-3 protein.

FIG. 10 shows W365-G14 and W365-G15 blocked the binding of PD-L1 to PD-1 expressing cells.

FIG. 11 shows W365-G14 and W365-G15 blocked the binding of LAG-3 to MHC-II.

FIG. 12 shows W365-G14 and W365-G15 enhanced NFAT pathways in PD-1 expressing Jurkat cells.

FIG. 13 shows W365-G14 and W365-G15 enhanced IL-2 pathways in LAG-3 expressing Jurkat cells.

FIG. 14 shows W365-G15 enhanced NFAT pathways in PD-1 and LAG-3 expressing Jurkat cells.

FIG. 15A shows W365-G15 enhanced IL-2 production in MLR assay.

FIG. 15B shows W365-G15 enhanced IFN-γ production in MLR assay.

FIG. 16 shows W365-G15 enhanced IL-2 production of PBMC stimulated with SEB.

FIG. 17A shows W365-G15 was stable in fresh human serum for up to 14 days.

FIG. 17B shows W365-G14 was stable in fresh human serum for up to 14 days.

FIG. 18A shows W365-G15 inhibited the growth of B16F10 tumor in human PD-1/LAG-3 knock-in transgenic mouse.

FIG. 18B shows the weight of human PD-1/LAG-3 knock-in transgenic mice carrying the B16F10 tumor over time after treatment with W365-G15.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, Chothia, AbM or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antibody molecule” as used herein refers to an antigen-binding protein or polypeptide comprising at least one antibody fragment (such as CDR, and/or variable region sequence). An antibody molecule includes, for example, a monoclonal antibody, an antibody fragment or domain, a fusion protein comprising an antibody fragment or domain, a polypeptide complex comprising an antibody fragment or domain, and so on.

The term “bivalent” as used herein refers to an antibody or an antigen-binding domain having two antigen-binding sites; the term “monovalent” refers to an antibody or an antigen-binding domain having only one single antigen-binding site; and the term “multivalent” refers to an antibody or an antigen-binding domain having multiple antigen-binding sites. In some embodiments, the antibody or antigen-binding domain thereof is bivalent.

The term “antigen-binding domain” (e.g. LAG-3-binding domain or PD-1-binding domain) as used herein refers to an antibody fragment formed from a portion of an antibody comprising one or more CDRs, or any other antibody fragment that binds to an antigen but does not comprise an intact native antibody structure. Examples of antigen-binding domain include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding domain is capable of binding to the same antigen to which the parent antibody binds. In certain embodiments, an antigen-binding domain may comprise one or more CDRs from a particular human antibody grafted to a framework region from one or more different human antibodies. For more and detailed formats of antigen-binding domain are described in Spiess et al, 2015 (Supra), and Brinkman et al., mAbs, 9(2), pp. 182-212 (2017), which are incorporated herein by entirety reference.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

A “fragment difficult (Fd)” with regard to an antibody refers to the amino-terminal half of the heavy chain fragment that can be combined with the light chain to form a Fab. For example, Fd fragment may consists of the VH and CH1 domains

“Fv” with regard to an antibody refers to the smallest fragment of the antibody to bear the complete antigen-binding site. An Fv fragment consists of the variable region of a single light chain bound to the variable region of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

“ScFab” refers to a fusion polypeptide with a Fd linked to a light chain via a polypeptide linker, resulting in the formation of a single chain Fab fragment (scFab).

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

“Appended IgG” refers to a fusion protein with a Fab arm fused to an IgG to form the format of bispecific (Fab)₂-Fc. It can form a “IgG-Fab” or a “Fab-IgG”, with a Fab fused to the C-terminus or N-terminus of an IgG molecule with or without a connector. In certain embodiments, the appended IgG can be further modified to a format of IgG-Fab₄ (see, Brinkman et al., 2017, Supra).

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as antibody-dependent cell-mediated cytotoxicity (ADCC), and complement dependent cytotoxicity (CDC), but does not function in antigen binding.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “chimeric” as used herein, means an antibody or antigen-binding domain, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding domain comprises CDRs derived from non-human animals, FR regions derived from human, and when applicable, the constant regions derived from human.

The term “fully human” as used herein, with reference to antibody or antigen-binding domain, means that the antibody or the antigen-binding domain has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

The term “operably link” or “operably linked” refers to a juxtaposition, with or without a spacer or a linker or an intervening sequence, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. For another example, an antigen-binding domain can be operably linked to another antigen-binding domain with an intervening sequence there between, and such intervening sequence can be a spacer or can comprise a much longer sequence such as a constant region of an antibody. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “fusion” or “fused” when used with respect to amino acid sequences (e.g. peptide, polypeptide or protein) refers to combination of two or more amino acid sequences, for example by chemical bonding or recombinant means, into a single amino acid sequence which does not exist naturally. A fusion amino acid sequence may be produced by genetic recombination of two encoding polynucleotide sequences, and can be expressed by a method of introducing a construct containing the recombinant polynucleotides into a host cell.

An “antigen” as used herein refers to a compound, composition, peptide, polypeptide, protein or substance that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

“LAG-3” (or “Lag3” or “Lag-3”) as used herein, refers to the lymphocyte-activation gene 3 derived from any vertebrate source, including mammals such as primates (e.g. humans, monkeys) and rodents (e.g., mice and rats). Exemplary sequence of human LAG-3 includes Homo sapiens (human) LAG-3 protein (NCBI Ref Seq No. CAA73914.1) (partial). Exemplary sequence of LAG-3 includes Rattus norvegicus (Rat) LAG-3 protein (NCBI RefSeq No. AAP57397.1).

The term “LAG-3” as used herein is intended to encompass any form of LAG-3, for example, 1) native unprocessed LAG-3 molecule, “full-length” LAG-3 chain or naturally occurring variants of LAG-3, including, for example, splice variants or allelic variants; 2) any form of LAG-3 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of LAG-3 subunit generated through recombinant method.

The term “anti-LAG-3 antibody”, “anti-LAG-3 binding domain” or “LAG-3-binding domain” refers to an antibody or antigen-binding domain that is capable of specific binding LAG-3 (e.g. human or monkey or mouse LAG-3).

“PD-1” as used herein refers programmed cell death protein, which belongs to the superfamily of immunoglobulin and functions as co-inhibitory receptor to negatively regulate the immune system. PD-1 is a member of the CD28/LAG-3 family, and has two known ligands including PD-L1 and PD-L2. Representative amino acid sequence of human PD-1 is disclosed under the NCBI accession number: NP_005009.2, and the representative nucleic acid sequence encoding the human PD-1 is shown under the NCBI accession number: NM_005018.2.

“PD-L1” as used herein refers to programmed cell death ligand 1 (PD-L1, see, for example, Freeman et al. (2000) J. Exp. Med. 192:1027). Representative amino acid sequence of human PD-L1 is disclosed under the NCBI accession number: NP_054862.1, and the representative nucleic acid sequence encoding the human PD-L1 is shown under the NCBI accession number: NM_014143.3. PD-L1 is expressed in placenta, spleen, lymph nodes, thymus, heart, fetal liver, and is also found on many tumor or cancer cells. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response.

“Anti-PD-1 antibody”, “anti-PD-1 binding domain” or “PD-1 binding domain” as used herein refers to an antibody or antigen-binding domain that is capable of specific binding to PD-1 (e.g. human or monkey PD-1) with an affinity which is sufficient to provide for diagnostic and/or therapeutic use.

The term “specific binding” or “specifically binds” as used herein refers to a non-random binding reaction between two molecules, such as for example between an antibody and an antigen. In certain embodiments, the antibody molecules or antigen-binding domains provided herein specifically bind to human PD-1 and/or human LAG-3 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹ M, ≤3×10⁻⁹ M, ≤2×10⁻⁹ M, ≤10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The ability to “block binding” or “compete for the same epitope” as used herein refers to the ability of an antibody or antigen-binding domain to inhibit the binding interaction between two molecules (e.g. human LAG-3 and an anti-LAG-3 antibody, human PD-1 and an anti-PD-1 antibody) to any detectable degree. In certain embodiments, an antibody or antigen-binding domain that blocks binding between two molecules inhibits the binding interaction between the two molecules by at least 85%, or at least 90%. In certain embodiments, this inhibition may be greater than 85%, or greater than 90%.

The term “epitope” as used herein refers to the specific group of atoms or amino acids on an antigen to which an antibody binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation. Two antibodies may bind the same or a closely related epitope within an antigen if they exhibit competitive binding for the antigen. For example, if an antibody or antigen-binding domain blocks binding of a reference antibody to the antigen by at least 85%, or at least 90%, or at least 95%, then the antibody or antigen-binding domain may be considered to bind the same/closely related epitope as the reference antibody.

The antibody names as used herein may include one or more suffix symbols which usually indicates the type of the antibody or particular modifications made to the antibody. For example, “uIgG4” means an antibody with human constant region of IgG4 isotype, “hAb” or “uAb” means human antibody, “K” means Kappa light chain, “L” means Lambda light chain, “SP” means an antibody having S228P mutation in human IgG4.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “homolog” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector.

The phrase “host cell” as used herein refers to a cell into which an exogenous polynucleotide and/or a vector has been introduced.

A “LAG-3-related” disease or condition as used herein refers to any disease or condition caused by, exacerbated by, or otherwise linked to increased or decreased expression or activities of LAG-3. In some embodiments, the LAG-3 related condition is immune-related disorder, such as, for example, cancer or infectious disease.

A “PD-1-related” disease or condition as used herein refers to any condition that is caused by, exacerbated by, or otherwise linked to increased or decreased expression or activities of PD-1 (e.g. a human PD-1).

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer. In certain embodiments, the cancer is selected from a lymphoma, Hodgkin's lymphoma, non-Hodgkin's lymphoma and B-cell lymphoma.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

A. Bispecific Antibody Molecule

In one aspect, the present disclosure provides herein a bispecific antibody molecule. The term “bispecific” as used herein means that, there are at least two antigen-binding domains (i.e. could be dual specific or multispecific), each of which is capable of specifically binding to a different epitope. The bispecific antibody molecule provided herein comprises a LAG-3-binding domain and a PD-1-binding domain, the LAG-3-binding domain comprises one independently selected from the group consisting of: a Fab and a scFv; and the PD-1-binding domain comprises one independently selected from the group consisting of: a Fab and a scFv.

i. LAG-3-Binding Domain

In certain embodiments, the LAG-3-binding domain comprises one or more (e.g. 1, 2, 3, 4, 5, or 6) CDR sequences of an anti-LAG-3 antibody of W3395-3.40.19.

“W3395-3.40.19” as used herein refers to a fully human antibody that comprises a heavy chain variable region of SEQ ID NO: 7, and a light chain variable region of SEQ ID NO: 8.

In certain embodiments, the LAG-3-binding domain comprises a heavy chain variable region comprising a heavy chain CDR1 comprising SEQ ID NO: 1, a heavy chain CDR2 comprising SEQ ID NO: 2, and a heavy chain CDR3 comprising SEQ ID NO: 3; and/or a light chain variable region comprising a light chain CDR1 comprising SEQ ID NO: 4, a light chain CDR2 comprising SEQ ID NO: 5, and a light chain CDR3 comprising SEQ ID NO: 6.

Table 1 shows the CDR sequences of the anti-LAG-3 antibody. The heavy chain and light chain variable region sequences are also provided below in Table 2 and Table 3.

TABLE 1

| | | CDR1 | CDR2 | CDR3 |
|---|---|---|---|---|
| W3395- | HCDR | SEQ ID NO: | SEQ ID NO: | SEQ ID NO: |
| 3.40.19 | | 1 | 2 | 3 |
| | | GDSISSTSYYWG | SFYYSGST | MQLWSYDVDV |
| | | | YYNPSLKS | |
| | LCDR | SEQ ID NO: | SEQ ID NO: | SEQ ID NO: |
| | | 4 | 5 | 6 |
| | | TGTSSDVGGYD | DVSERPS | SSYTSTTTLVV |
| | | YVA | | |

TABLE 2

| | VH | VL |
|---|---|---|
| W3395- | SEQ ID NO: 7 | SEQ ID NO: 8 |
| 3.40.19 | QLQLQESGPGLVKPSETLSL | QSALTQPASVSGSPGQSITIS |
| | TCTVSGDSISSTSYYWGWIR | CTGTSSDVGGYDYVAWYQQHP |
| | QPPGKGLEWIGSFYYSGSTY | GKVPKLMIYDVSERPSGVSNR |
| | YNPSLKSRVTISVDTSKNQF | FSGSKSGNTASLTISGLQAED |
| | SLKLNSVTAADTAVYYCARM | EADYYCSSYTSTTTLVVFGGG |
| | QLWSYDVDVWGQGTTVTVSS | TKLSVL |

TABLE 3

| | VHnu | VLnu |
|---|---|---|
| W3395-3.40.19 | SEQ ID NO: 9 | SEQ ID NO: 10 |
| | cagctgcagctgcaggagtcgggcccagg | cagtctgccctgactcaacctgcctccgtgtctggg |
| | actggtgaagccttcggagaccctgtccctc | tctcctggacagtcgatcaccatctcctgcactgga |
| | acctgcactgtctctggtgactccatcagcag | accagcagtgacgttggtgggtatgactatgtcgc |
| | tactagttactactggggctggatccgccag | ctggtaccaacaacacccaggcaaagtccccaaa |
| | cccccagggaaggggctggagtggattgg | ctcatgatttatgatgtcagtgagcggccctcaggg |
| | gagtttctattatagtgggagcacctactaca | gtttctaatcgcttctctggctccaagtctggcaaca |
| | acccgtccctcaagagtcgagtcaccatttc | cggcctccctgaccatctctgggctccaggctgag |
| | cgtagacacgtccaagaaccagttctccctg | gacgaggctgattattactgcagctcatatacaagc |
| | aagctgaactctgtgaccgccgcagacacg | accaccactctcgttgtgttcggcggagggaccaa |
| | gctgtgtattactgtgcgaggatgcagctatg | gctgtccgtcctg |
| | gtcgtacgatgtggacgtctggggccaagg | |
| | gaccacggtcaccgtctcctca | |

CDRs are known to be responsible for antigen binding, however, it has been found that not all of the 6 CDRs are indispensable or unchangeable. In other words, it is possible to replace or change or modify one or more CDRs provided herein for LAG-3-binding domains, yet substantially retain the specific binding affinity to LAG-3.

In certain embodiments, the LAG-3-binding domains provided herein comprise a heavy chain CDR3 sequence of the anti-LAG-3 antibody W3395-3.40.19. In certain embodiments, the anti-LAG-3 antibodies and the antigen-binding fragments provided herein comprise a heavy chain CDR3 sequence comprising the sequence of SEQ ID NO: 3.

Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the LAG-3-binding domains provided herein comprise any suitable framework region (FR) sequences, as long as the antigen-binding domains can specifically bind to LAG-3. In certain embodiments, the CDR sequences of W3395-3.40.19 are obtained from rat antibodies, but they can be grafted to any suitable FR sequences of any suitable species such as mouse, human, rat, rabbit, among others, using suitable methods known in the art such as recombinant techniques.

In certain embodiments, the anti-LAG-3 antibodies and the antigen-binding fragments thereof provided herein are fully human. Fully human antibodies can be prepared using recombinant methods. For example, transgenic animal such as a mouse can be made to carry transgenes or transchromosomes of human immunoglobulin genes, and therefore capable of producing fully human antibodies after immunization with proper antigen. Fully human antibodies can be isolated from such transgenic animal, or alternatively, can be made by hybridoma technology by fusing the spleen cells of the transgenic animal with an immortal cell line to generate hybridoma cells secreting the fully human antibodies. Exemplary transgenic animals include, without limitation, OmniRat, whose endogenous expression of rat immunoglobulin genes are inactivated and at the same time engineered to contain functional recombinant human immunoglobulin loci; OmniMouse, whose endogenous expression of mouse immunoglobulin genes are inactivated and at the same time engineered to contain recombinant human immunoglobulin loci having J-locus deletion and a C-kappa mutation; OmniFlic, which is a transgenic rat whose endogenous expression of rat immunoglobulin genes are inactivated and at the same time engineered to contain recombinant human immunoglobulin loci having a single common, rearranged VkJk light chain and functional heavy chain. Detailed information can be further found at: Osborn M. et al, Journal of Immunology, 2013, 190: 1481-90; Ma B. et al, Journal of Immunological Methods 400-401 (2013) 78-86; Geurts A. et al, Science, 2009, 325:433; U.S. Pat. No. 8,907,157; EP patent 2152880B1; EP patent 2336329B1, all of which are incorporated herein by reference to its entirety. Other suitable transgenic animals can also be used, for example, HuMab mice (see, for details, Lonberg, N. et al. Nature 368(6474): 856 859 (1994)), Xeno-Mouse (Mendez et al. Nat Genet., 1997, 15:146-156), TransChromo Mouse (Ishida et al. Cloning Stem Cells, 2002, 4:91-102) and VelocImmune Mouse (Murphy et al. Proc Natl Acad Sci USA, 2014, 111:5153-5158), Kymouse (Lee et al. Nat Biotechnol, 2014, 32:356-363), and transgenic rabbit (Flisikowska et al. PLoS One, 2011, 6:e21045).

In certain embodiments, the LAG-3-binding domains provided herein comprise a heavy chain variable domain sequence of SEQ ID NO: 7. In certain embodiments, LAG-3-binding domains provided herein comprise a light chain variable domain sequence of SEQ ID NO: 8.

In some embodiments, the LAG-3-binding domains provided herein comprise all or a portion of the heavy chain variable domain and/or all or a portion of the light chain variable domain. In one embodiment, the LAG-3-binding domains provided herein are a single domain antibody which consists of all or a portion of the heavy chain variable domain provided herein. More information of such a single domain antibody is available in the art (see, e.g., U.S. Pat. No. 6,248,516).

ii. PD-1-Binding Domain

In certain embodiments, the PD-1-binding domain is capable of specifically binding to PD-1 (such as human PD-1), and comprises one independently selected from the group consisting of: a Fab and a scFv.

In certain embodiments, the PD-1-binding domain comprises one or more (e.g. 1, 2, 3, 4, 5, or 6) CDR sequences of an anti-PD-1 antibody W3055-1.153.7.

“W3055-1.153.7” as used herein refers to a fully human monoclonal antibody having a heavy chain variable region of SEQ ID NO: 17, and a kappa light chain variable region of SEQ ID NO: 18.

In certain embodiments, the PD-1-binding domain comprises a heavy chain variable region comprising a heavy chain CDR1 comprising SEQ ID NO: 11, a heavy chain CDR2 comprising SEQ ID NO: 12, and a heavy chain CDR3 comprising SEQ ID NO: 13, and/or a light chain variable region comprising a light chain CDR1 comprising SEQ ID NO: 14, a light chain CDR2 comprising SEQ ID NO: 15, and a light chain CDR3 comprising SEQ ID NO: 16.

Table 4 shows the CDR sequences of the anti-PD-1 antibody. The heavy chain and light chain variable region sequences are also provided below in Table 5 and Table 6.

TABLE 4
CDR amino acid sequences

| | | CDR1 | CDR2 | CDR3 |
|---|---|---|---|---|
| W3055-1.153.7 | HCDR | SEQ ID NO: | SEQ ID NO: | SEQ ID NO: |
| | | 11 | 12 | 13 |
| | | GFTFSSHAMS | TITGGGGSIYY | NRAGEGYFDY |
| | | | ADSVKG | |
| | LCDR | SEQ ID NO: | SEQ ID NO: | SEQ ID NO: |
| | | 14 | 15 | 16 |
| | | GGDNIGNKDVH | RDSNRPS | QVWDSIWV |

TABLE 5
Variable region amino acid sequences

| | VH | VL |
|---|---|---|
| W3055- | SEQ ID NO: 17 | SEQ ID NO: 18 |
| 1.153.7 | EVQLLESGGGLVQPGGSLR | SYELTQPLSVSVALGQTARITC |
| | LSCAASGFTFSSHAMSWVR | GGDNIGNKDVHWYQQKPGQAPV |
| | QAPGKGLEWVSTITGGGGS | LVIYRDSNRPSGIPEGFSGSNS |
| | IYYADSVKGRFTISRDNSK | GNTATLTISRAQAGDEADYYCQ |
| | NTLYLQMNSLRAEDTAVYY | VWDSIWVFGGGTKLTVL |
| | CAKNRAGEGYFDYWGQGTL | |
| | VTVSS | |

TABLE 6
Variable region nucleotide sequences

| | VHnu | VLnu |
|---|---|---|
| W3055-1.153.7 | SEQ ID NO: 19 | SEQ ID NO: 20 |
| | gaggtgcagctgttggagtctgggggaggc | tcctatgagctgactcagccactctcagtgtcagtg |
| | ttggtacagcctggggggtccctgagactgt | gccctgggacagacggccaggattacctgtggg |
| | cctgcgcagcctctggattcacctttagcagc | ggagacaacattggaaataaagatgtgcactggta |
| | catgccatgagctgggtccgccaggctcca | ccagcagaagccaggccaggcccctgtgctggtc |
| | gggaaggggctggagtgggtctcaactatt | atctatagggatagcaaccggccctctgggatccc |
| | actggtggtggtggtagcatatactacgcag | tgagggattctctggctccaactcggggaacacg |
| | actccgtgaagggccggttcaccatctccag | gccaccctgaccatcagcagagcccaagccggg |
| | agacaattccaagaacacgctgtatctgcaa | gatgaggctgactattactgtcaggtgtgggacagc |
| | atgaacagcctgagagccgaggacacggc | atagggtgttcggcggagggaccaagctgaccgtccta |
| | cgtatattattgtgcgaaaaaccgcgctggg | |
| | gagggttactttgactactggggccagggaa | |
| | ccctggtcaccgtctcctca | |

CDRs are known to be responsible for antigen binding, however, it has been found that not all of the 6 CDRs are indispensable or unchangeable. In other words, it is possible to replace or change or modify one or more CDRs provided herein for PD-1-binding domains, yet substantially retain the specific binding affinity to PD-1 (e.g. human PD-1).

In certain embodiments, the PD-1-binding domains provided herein comprise SEQ ID NO: 13 (i.e. a heavy chain CDR3 sequence of anti-PD-1 antibody W3055-1.153.7).

In certain embodiments, the PD-1-binding domains provided herein are fully human. For example, the PD-1-binding domains of W3055-1.153.7 is fully human.

In certain embodiments, the PD-1-binding domains provided herein comprise a heavy chain variable domain sequence comprising SEQ ID NO: 17. In certain embodiments, PD-1-binding domains provided herein comprise a light chain variable domain sequence comprising SEQ ID NO: 18.

In some embodiments, the PD-1-binding domains provided herein comprise all or a portion of the heavy chain variable domain and/or all or a portion of the light chain variable domain. In one embodiment, the PD-1-binding domains provided herein are a single domain antibody which consists of all or a portion of the heavy chain variable domain provided herein. More information of such a single domain antibody is available in the art (see, e.g., U.S. Pat. No. 6,248,516).

iii. Bispecific Antibody Molecule

In certain embodiments, the bispecific antibody molecules provided herein comprises an LAG-3-binding domain comprising one or more (e.g. 1, 2, 3, 4, 5, or 6) CDR sequences selected from SEQ ID NOs: 1-6 (i.e. derived from W3395-3.40.19), and a PD-1-binding domain comprising one or more (e.g. 1, 2, 3, 4, 5, or 6) CDR sequences selected from SEQ ID Nos: 11-16 (i.e. derived from W3055-1.153.7), and the LAG-3-binding domain comprises one independently selected from the group consisting of: a Fab and a scFv, the PD-1-binding domain comprises one independently selected from the group consisting of: a Fab and a scFv.

In certain embodiments, the LAG-3-binding domain comprises a heavy chain variable region comprising the sequence of SEQ ID NO: 7, or a homologous sequence thereof having at least 80% sequence identity yet retaining specific binding affinity to LAG-3 (e.g. human LAG-3), and/or a light chain variable region comprising the sequence of SEQ ID NO: 8, or a homologous sequence thereof having at least 80% sequence identity yet retaining specific binding affinity to LAG-3 (e.g. human LAG-3).

In certain embodiments, the PD-1 binding domain comprises a heavy chain variable region comprising the sequence of SEQ ID NO: 17 or a homologous sequence thereof having at least 80% sequence identity yet retaining specific binding affinity to PD-1 (e.g. human PD-1), and/or a light chain variable region comprising the sequence of SEQ ID NO: 18 or a homologous sequence thereof having at least 80% sequence identity yet retaining specific binding affinity to PD-1 (e.g. human PD-1).

In certain embodiments, the LAG-3-binding domain comprises a heavy chain variable region comprising the sequence of SEQ ID NO: 7 and a light chain variable region comprising the sequence of SEQ ID NO: 8 (derived from W3395-3.40.19), and the PD-1 binding domain comprises a heavy chain variable region comprising the sequence of SEQ ID NO: 17 and a light chain variable region comprising the sequence of SEQ ID NO: 18 (derived from W3055-1.153.7) (such bispecific antibody molecules are also referred to as “W365B” herein).

The LAG-3-binding domains and/or the PD-1-binding domains provided herein comprise one independently selected from the group consisting: a Fab and a scFv.

Various techniques can be used for the production of such antigen-binding domains. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. coli (e.g. for Fab, Fv and ScFv antibody fragments), screening from a phase display library as discussed above (e.g. for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the LAG-3-binding domain and/or the PD-1-binding domain is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck). An scFv can comprise from a VH linked directly or via a peptide linker to a VL. In certain embodiments, the VH can be at the N-terminus and the VL can be at the C terminus of the scFv. In certain embodiments, the VL can be at the N-terminus and the VH can be at the C terminus of the scFv.

In certain embodiments, the PD-1-binding domain comprises or is a scFv comprising a heavy chain variable region (VH) comprising the sequence of SEQ ID NO: 17 (W3055-1.153.7 VH) linked to a light chain variable region (VL) comprising the sequence of SEQ ID NO: 18 (W3055-1.153.7 VL) via a peptide linker. In certain embodiments, the LAG-3-binding domain comprises or is a scFv comprising a heavy chain variable region (VH) comprising the sequence of SEQ ID NO: 7 (W3395-3.40.19 VH) linked to a light chain variable region (VL) comprising the sequence of SEQ ID NO: 8 (W3395-3.40.19 VL) via a peptide linker.

The peptide linker can comprise a single or repeated sequences composed of threonine/serine and glycine, such as TGGGG (SEQ ID NO: 43), GGGGS (SEQ ID NO: 39), GGGGSGGGGS (SEQ ID NO: 40), GGGGSGGGGSGGGGS (SEQ ID NO: 41) or SGGGG (SEQ ID NO: 44) or its tandem repeats (e.g. 2, 3, 4, or more repeats). In certain embodiments, the peptide linker comprises GGGGSGGGGSGGGGSGGGGS (SEQ ID NO: 42).

In certain embodiments, the LAG-3-binding domain comprises or is a scFv comprising a VH comprising the sequence of SEQ ID NO: 7 (W3395-3.40.19VH) linked to the N-terminus of a VL comprising the sequence of sequence of SEQ ID NO: 8 (W3395-3.40.19 VL) via a peptide linker. In certain embodiments, the peptide linker comprises or is SEQ ID NO: 41. In certain embodiments, the LAG-3-binding domain comprises a scFv comprising SEQ ID NO: 38.

In certain embodiments, the LAG-3-binding domain and/or the PD-1-binding domain comprises or is a Fab. In certain embodiments, the PD-1-binding domain is a Fab comprising a heavy chain variable region comprising SEQ ID NO: 17 (W3055-1.153.7 VH) and a light chain variable region comprising SEQ ID NO: 18 (W3055-1.153.7 VL). In certain embodiments, the LAG-3-binding domain comprises or is a Fab comprising a heavy chain variable region SEQ ID NO: 7 (W3395-3.40.19 VH) and a light chain variable region SEQ ID NO: 8 (W3395-3.40.19 VL). The heavy chain variable region and the light chain variable region can be disulfidely bonded. The term “disulfidely bonded” refers to linkage via one or more disulfide bond (optionally in addition to another bond). A disulfide bond can be formed between, for example, one cysteine residue of an antibody heavy chain and another cysteine residue of the light chain.

In certain embodiments, the LAG-3-binding and/or the PD-1-binding domains are multivalent, such as bivalent, trivalent, tetravalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. A bivalent molecule can be monospecific if the two binding sites are both for specific binding of the same antigen or the same epitope. Similarly, a trivalent molecule can be bispecific, for example, when two binding sites are monospecific for a first antigen (or epitope) and the third binding site is specific for a second antigen (or epitope). In certain embodiments, the LAG-3-binding and/or the PD-1-binding domains in the bispecific antibody molecule provided herein can be bivalent, trivalent, or tetravalent, with at least two binding sites specific for the same antigen or epitope. This, in certain embodiments, provides for stronger binding to the antigen or the epitope than a monovalent counterpart. In certain embodiments, in a bivalent antigen-binding moiety, the first valent of binding site and the second valent of binding site are structurally identical (i.e. having the same sequences), or structurally different (i.e. having different sequences albeit with the same specificity). In certain embodiments, LAG-3-binding and/or the PD-1-binding domains comprises two or more antigen binding sites (e.g. scFv or Fab) operably linked together, with or without a spacer.

In certain embodiments, the LAG-3-binding domain is operably linked to the N terminus or the C terminus of the PD-1-binding domain. In certain embodiments, the PD-1-binding domain is operably linked to the N terminus or the C terminus of the LAG-3-binding domain.

The operable linkage can be a direct chemical bond linkage or linkage via a spacer or via an intervening sequence. The term “spacer” as used herein refers to an artificial amino acid sequence having 1, 2, 3, 4 or 5 amino acid residues, or a length of between 5 and 15, 20, 30, 50 or more amino acid residues, joined by peptide bonds and are used to link one or more binding domains, such as a scFv and a Fab or an IgG. The spacer can be identical to or different from the peptide linker in the scFv. In certain embodiment, the spacer comprises 1, 2, 3, 4 or more sequential or tandem repeats of SEQ ID NOs: 39, 40 and 42. In certain embodiments, the spacer comprises GGGGS (SEQ ID NO: 39). In certain embodiments, the spacer comprises GGGGSGGGGS (SEQ ID NO: 40), GGGGSGGGGSGGGGS (SEQ ID NO: 41), GGGGSGGGGSGGGGSGGGGS (SEQ ID NO: 42). The intervening sequence as used herein can be any amino acid sequence located between the LAG-3-binding domain and the PD-1-binding domain, as long as both the LAG-3-binding domain and the PD-1-binding domain are capable of binding to its respective antigen. In an illustrative example, the intervening sequence can comprise a heavy chain constant region, or a light chain constant region.

In certain embodiments, the LAG-3-binding domain comprises a scFv and the PD-1-binding domain comprises a Fab or an IgG. In certain embodiments, the LAG-3-binding scFv can be operably linked to the N terminus or the C-terminus of the heavy chain of the anti-PD-1 binding Fab or IgG (e.g. the C-terminus of the heavy chain constant region following the PD-1-binding Fab), or to the N terminus or the C-terminus of the light chain of the anti-PD-1 binding Fab or IgG, or any combination thereof, and vice versa.

In an illustrative example, the bispecific antibody molecule can comprise a heavy chain in the format of: VH(anti-PD-1)-CH1-Hinge-CH2-CH3-spacer-scFv(anti-LAG-3) or scFv (anti-LAG-3)-spacer-VH(anti-PD-1)-CH1-Hinge-CH2-CH3, and a light chain in the format of: VL(anti-PD-1)-CL. As used herein, VH(anti-PD-1) and VL(anti-PD-1) refer respectively to the heavy and light chain variable domain of the PD-1 binding domain; scFv(anti-LAG-3) refers to scFv of the LAG-3-binding domain, CL refers to the light chain constant region; and CH1-Hinge-CH2-CH3 are collectively heavy chain constant region.

In another illustrative example, the bispecific antibody molecule can comprise a light chain in the format of: scFv (anti-LAG-3)-spacer-VL(anti-PD-1)-CL or VL(anti-PD-1)-CL-spacer-scFv(anti-LAG-3), and a heavy chain in the format of: VH(anti-PD-1)-CH1-Hinge-CH2-CH3, by the same token.

In certain embodiments, when the PD-1-binding domain is a scFv and the LAG-3-binding domain is a Fab or an IgG, the PD-1-binding domain scFv can be operably linked to the N terminus or the C-terminus of the heavy chain of the anti-LAG-3 Fab or IgG, or to the N terminus or the C-terminus of the light chain of the anti-LAG-3 Fab or IgG, or any combination thereof, and vice versa.

In an illustrative example, the bispecific antibody molecule can comprise a heavy chain in the format of: VH (anti-LAG-3)-CH1-Hinge-CH2-CH3-spacer-scFv(anti-PD1) or scFv(anti-PD1)-spacer-VH(anti-LAG-3)-CH1-Hinge-CH2-CH3, and a light chain in the format of: VL(anti-LAG-3)-CL. In another illustrative example, the bispecific antibody molecule can comprise a light chain in the format of: scFv (anti-PD-1)-spacer-VL(anti-LAG-3)-CL or VL(anti-LAG-3)-CL-spacer-scFv (anti-PD-1), and a heavy chain in the format of: VH(anti-LAG-3)-CH1-Hinge-CH2-CH3, by the same token.

In the bispecific antibody molecule provided herein, the LAG-3-binding domain may be monovalent (i.e. one scFv or Fab) or multivalent (e.g. more than one scFv or Fab), and/or the PD-1-binding domain may be monovalent or multivalent.

In certain embodiments, the bispecific antibody molecule comprise a heavy chain in the format of: VH(anti-PD-1)-CH1-Hinge-CH2-CH3, and a light chain in the format of VL(anti-PD-1)-CL-spacer-scFv(anti-LAG-3), wherein the VH(anti-PD-1) comprises a sequence of SEQ ID NO: 17, the VL (anti-PD-1) comprises an amino acid sequence of SEQ ID NO: 18, and the scFv (anti-LAG-3) comprises the sequence of SEQ ID NO: 38. In certain embodiments, the spacer comprises the sequence of SEQ ID NO: 40. In certain embodiments the heavy chain constant region is of human IgG4 isotype, and optionally contains mutations of S228P and/or L235E. In certain embodiments, the heavy chain constant region comprises the sequence of SEQ ID NO: 35 or 37. In certain embodiments, the light chain constant region comprises the sequence of SEQ ID NO: 36. In certain embodiments, the bispecific antibody molecule comprises a heavy chain comprising the amino acid sequence of SEQ ID NO: 31 and a light chain comprising the amino acid sequence of SEQ ID NO: 32.

In certain embodiments, the bispecific antibody molecule comprise a heavy chain in the format of: VH(anti-PD-1)-CH1-Hinge-CH2-CH3-spacer-scFv(anti-LAG-3), and a light chain in the format of VL(anti-PD-1)-CL, wherein the VH(anti-PD-1) comprises a sequence of SEQ ID NO: 17, the scFv (anti-LAG-3) comprises the sequence of SEQ ID NO: 38, and the VL (anti-PD-1) comprises an amino acid sequence of SEQ ID NO: 18. In certain embodiments, the spacer comprises the sequence of SEQ ID NO: 42. In certain embodiments the heavy chain constant region is of human IgG4 isotype, and optionally contains mutations of S228P and/or L235E. In certain embodiments, the heavy chain constant region comprises the sequence of SEQ ID NO: 35 or 37. In certain embodiments, the light chain constant region comprises the sequence of SEQ ID NO: 36. In certain embodiments, the bispecific antibody molecule comprises a heavy chain comprising the amino acid sequence of SEQ ID NO: 33 and a light chain comprising the amino acid sequence of SEQ ID NO: 34.

Tables 7 and 8 show the combination of heavy chain and light chain sequences of the bispecific antibody molecules of W365B (specifically W365-G14 and W365-G15).

TABLE 7

| W365- | Heavy chain | VH(Anti-PD-1) | CH (IgG4S228P) | | | | |
|---|---|---|---|---|---|---|---|
| U6T1.G14- | (SEQ ID NO: 31) | (3055_1.153.7) | SEQ ID NO: 35 | | | | |
| 1.uIgG4.SP | | SEQ ID NO: 17 | | | | | |
| (W365-G14) | Light chain | VL(Anti-PD-1) | CL | Spacer | scFv(Anti-LAG-3) (SEQ ID NO: 38) | | |
| | (SEQ ID NO: 32) | (3055_1.153.7) | SEQ ID NO: 36 | SEQ ID NO: 40 | VH(Anti-LAG-3) | Linker | VL(Anti-LAG-3) |
| | | SEQ ID NO: 18 | | | (3395-3.40.19) | SEQ ID NO: 41 | (3395-3.40.19) |
| | | | | | SEQ ID NO: 7 | | SEQ ID NO: 8 |
| W365- | Heavy chain | VH(Anti-PD-1) | CH | Spacer | Anti-LAG-3 scFv (3395-3.40.19) (SEQ ID NO: 38) | | |
| U6T1.G15- | (SEQ ID NO: 33) | (3055_1.153.7) | (IgG4S228P) | SEQ ID NO: 42 | VH(Anti-LAG-3) | Linker | VL(Anti-LAG-3) |
| 1.uIgG4.SP | | SEQ ID NO: 17 | SEQ ID NO: 37 | | (3395-3.40.19) | SEQ ID NO: 41 | (3395-3.40.19) |
| (W365-G15) | | | | | SEQ ID NO: 7 | | SEQ ID NO: 8 |
| | Light chain | VL(Anti-PD-1) | CL | | | | |
| | (SEQ ID NO: 34) | (3055_1.153.7) | SEQ ID NO: 36 | | | | |
| | | SEQ ID NO: 18 | | | | | |

“CL” refers to light chain constant region;
“CH” refers to heavy chain constant region;
“VL” refers to light chain variable region;
“VH” refers to heavy chain variable region;
“Anti-PD-1” refers to anti-PD-1 antibody, in particular, the sequence provided in the table is the sequence derived from anti-PD-1 antibody W3055_1.153.7.
“Anti-LAG-3” refers to anti-LAG-3 antibody, in particular, the sequence provided in the table is the sequence derived from anti-LAG-3 antibody W3395-3.40.19.

TABLE 8

| | |
|---|---|
| W365-G14: | SEQ ID NO: 31 |
| Heavy Chain | EVQLLESGGGLVQPGGSLRLSCAASGFTFSSHAMSWVRQAPGK |
| | GLEWVSTITGGGGSIYYADSVKGRFTISRDNSKNTLYLQMNSLR |
| | AEDTAVYYCAKNRAGEGYFDYWGQGTLVTVSSASTKGPSVFPL |
| | APCSRSTSESTAALGCLVKDYFPEPVTVSWNSGALTSGVHTFPA |
| | VLQSSGLYSLSSVVTVPSSSLGTKTYTCNVDHKPSNTKVDKRVE |
| | SKYGPPCPPCPAPEFLGGPSVFLFPPKPKDTLMISRTPEVTCVVVD |
| | VSQEDPEVQFNWYVDGVEVHNAKTKPREEQFNSTYRVVSVLTV |
| | LHQDWLNGKEYKCKVSNKGLPSSIEKTISKAKGQPREPQVYTLP |
| | PSQEEMTKNQVSLTCLVKGFYPSDIAVEWESNGQPENNYKTTPP |
| | VLDSDGSFFLYSRLTVDKSRWQEGNVFSCSVMHEALHNHYTQK |
| | SLSLSLGK |
| W365-G14: | SEQ ID NO: 32 |
| Light Chain | SYELTQPLSVSVALGQTARITCGGDNIGNKDVHWYQQKPGQAP |
| | VLVIYRDSNRPSGIPEGFSGSNSGNTATLTISRAQAGDEADYYCQ |
| | VWDSIWVFGGGTKLTVLGQPKAAPSVTLFPPSSEELQANKATLV |
| | CLISDFYPGAVTVAWKADSSPVKAGVETTTPSKQSNNKYAASSY |
| | LSLTPEQWKSHKSYSCQVTHEGSTVEKTVAPTECSGGGGSGGG |
| | GSQLQLQESGPGLVKPSETLSLTCTVSGDSISSTSYYWGWIRQPP |
| | GKGLEWIGSFYYSGSTYYNPSLKSRVTISVDTSKNQFSLKLNSVT |
| | AADTAVYYCARMQLWSYDVDVWGQGTTVTVSSGGGGSGGGG |
| | SGGGGSQSALTQPASVSGSPGQSITISCTGTSSDVGGYDYVAWY |
| | QQHPGKVPKLMIYDVSERPSGVSNRFSGSKSGNTASLTISGLQAE |
| | DEADYYCSSYTSTTTLVVFGGGTKLSVL |
| W365-G15: | SEQ ID NO: 33 |
| Heavy Chain | EVQLLESGGGLVQPGGSLRLSCAASGFTFSSHAMSWVRQAPGK |
| | GLEWVSTITGGGGSIYYADSVKGRFTISRDNSKNTLYLQMNSLR |
| | AEDTAVYYCAKNRAGEGYFDYWGQGTLVTVSSASTKGPSVFPL |
| | APCSRSTSESTAALGCLVKDYFPEPVTVSWNSGALTSGVHTFPA |
| | VLQSSGLYSLSSVVTVPSSSLGTKTYTCNVDHKPSNTKVDKRVE |
| | SKYGPPCPPCPAPEFLGGPSVFLFPPKPKDTLMISRTPEVTCVVVD |
| | VSQEDPEVQFNWYVDGVEVHNAKTKPREEQFNSTYRVVSVLTV |
| | LHQDWLNGKEYKCKVSNKGLPSSIEKTISKAKGQPREPQVYTLP |
| | PSQEEMTKNQVSLTCLVKGFYPSDIAVEWESNGQPENNYKTTPP |
| | VLDSDGSFFLYSRLTVDKSRWQEGNVFSCSVMHEALHNHYTQK |
| | SLSLSLGGGGGSGGGGSGGGGSGGGGSQLQLQESGPGLVKPSET |
| | LSLTCTVSGDSISSTSYYWGWIRQPPGKGLEWIGSFYYSGSTYYN |
| | PSLKSRVTISVDTSKNQFSLKLNSVTAADTAVYYCARMQLWSY |
| | DVDVWGQGTTVTVSSGGGGSGGGGSGGGGSQSALTQPASVSGS |
| | PGQSITISCTGTSSDVGGYDYVAWYQQHPGKVPKLMIYDVSERP |
| | SGVSNRFSGSKSGNTASLTISGLQAEDEADYYCSSYTSTTTLVVF |
| | GGGTKLSVL |
| W365-G15: | SEQ ID NO: 34 |
| Light Chain | SYELTQPLSVSVALGQTARITCGGDNIGNKDVHWYQQKPGQAP |
| | VLVIYRDSNRPSGIPEGFSGSNSGNTATLTISRAQAGDEADYYCQ |
| | VWDSIWVFGGGTKLTVLGQPKAAPSVTLFPPSSEELQANKATLV |
| | CLISDFYPGAVTVAWKADSSPVKAGVETTTPSKQSNNKYAASSY |
| | LSLTPEQWKSHKSYSCQVTHEGSTVEKTVAPTECS |
| W365-G14: | SEQ ID NO: 35 |
| Heavy chain | ASTKGPSVFPLAPCSRSTSESTAALGCLVKDYFPEPVTVSWNSGA |
| constant region | LTSGVHTFPAVLQSSGLYSLSSVVTVPSSSLGTKTYTCNVDHKPS |
| (CH) | NTKVDKRVESKYGPPCPPCPAPEFLGGPSVFLFPPKPKDTLMISR |
| | TPEVTCVVVDVSQEDPEVQFNWYVDGVEVHNAKTKPREEQFNS |
| | TYRVVSVLTVLHQDWLNGKEYKCKVSNKGLPSSIEKTISKAKGQ |
| | PREPQVYTLPPSQEEMTKNQVSLTCLVKGFYPSDIAVEWESNGQ |
| | PENNYKTTPPVLDSDGSFFLYSRLTVDKSRWQEGNVFSCSVMHE |
| | ALHNHYTQKSLSLSLGK |
| W365-G14 or | SEQ ID NO: 36 |
| W365-G15: | GQPKAAPSVTLFPPSSEELQANKATLVCLISDFYPGAVTVAWKA |
| Light Chain | DSSPVKAGVETTTPSKQSNNKYAASSYLSLTPEQWKSHKSYSCQ |
| Constant Region | VTHEGSTVEKTVAPTECS |
| (CL) | |
| W365-G15: | SEQ ID NO: 37 |
| Heavy chain | ASTKGPSVFPLAPCSRSTSESTAALGCLVKDYFPEPVTVSWNSGA |
| constant region | LTSGVHTFPAVLQSSGLYSLSSVVTVPSSSLGTKTYTCNVDHKPS |
| (CH) | NTKVDKRVESKYGPPCPPCPAPEFLGGPSVFLFPPKPKDTLMISR |
| | TPEVTCVVVDVSQEDPEVQFNWYVDGVEVHNAKTKPREEQFNS |
| | TYRVVSVLTVLHQDWLNGKEYKCKVSNKGLPSSIEKTISKAKGQ |
| | PREPQVYTLPPSQEEMTKNQVSLTCLVKGFYPSDIAVEWESNGQ |
| | PENNYKTTPPVLDSDGSFFLYSRLTVDKSRWQEGNVFSCSVMHE |
| | ALHNHYTQKSLSLSLG |
| scFv(Anti-LAG-3) | SEQ ID NO: 38 |
| (derived from | QLQLQESGPGLVKPSETLSLTCTVSGDSISSTSYYWGWIRQPPGK |
| W3395-3.40.19, | GLEWIGSFYYSGSTYYNPSLKSRVTISVDTSKNQFSLKLNSVTAA |
| in the format of | DTAVYYCARMQLWSYDVDVWGQGTTVTVSSGGGGSGGGGSG |
| VH-(G4S)3-VL) | GGGSQSALTQPASVSGSPGQSITISCTGTSSDVGGYDYVAWYQQ |
| | HPGKVPKLMIYDVSERPSGVSNRFSGSKSGNTASLTISGLQAEDE |
| | ADYYCSSYTSTTTLVVFGGGTKLSVL |

In certain embodiments, the bispecific antibody molecules provided herein may further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

The bispecific antibody molecules provided herein can have a constant region of an immunoglobulin (Ig), optionally a human Ig, optionally a human IgG. The constant region can be in any suitable isotype. In certain embodiments, the bispecific antibody molecules provided herein comprises a constant region of IgG1 isotype, which could induce ADCC or CDC, or a constant region of IgG4 or IgG2 isotype, which has reduced or depleted effector function.

In some embodiments, the bispecific antibody molecules provided herein have reduced or depleted effector function. In some embodiments, the bispecific antibody molecules provided herein have a constant region of IgG4 isotype, which has reduced or depleted effector function. Effector functions such as ADCC and CDC can lead to cytotoxicity to cells expressing PD-1. Many cells such as T cells normally express PD-1. In order to avoid potential unwanted toxicity to those normal cells, certain embodiments of the antibodies and antigen-binding fragments provided herein can possess reduced or even depleted effector functions. Various assays are known to evaluate ADCC or CDC activities, for example, Fc receptor binding assay, C1q binding assay, and cell lysis assay, and can be readily selected by people in the art. Without wishing to be bound to theory, but it is believed that antibodies with reduced or depleted effector functions such as ADCC or CDC would cause no or minimal cytotoxicity to PD-1-expressing cells, for example those T cells, and therefore spare them from unwanted side effects, whereas in the meantime, blocking of PD-1 would boost immune system for the treatment of conditions such as cancer or chronic infection.

In certain embodiments, the bispecific antibody molecules provided herein have reduced side effects. For example, the bispecific antibody molecules provided herein can comprise at least one fully human antigen-binding domain and Fc region and therefore reduced immunogenicity than a humanized antibody counterpart.

B. Characterization of the Bispecific Antibody Molecule

In some embodiments, the bispecific antibody molecules provided herein are capable of specifically binding to both human PD-1 and human LAG-3. The bispecific antibody molecules provided herein retain the specific binding affinity to both PD-1 and LAG-3, in certain embodiments are at least comparable to, or even better than, the parent antibodies in that aspect.

In certain embodiments, the bispecific antibody molecules provided herein have a specific binding affinity to LAG-3 which is sufficient to provide for diagnostic and/or therapeutic use.

Binding of bispecific antibody molecules can also be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assay. In certain embodiments, the bispecific antibody molecules provided herein specifically bind to human PD-1 at an EC₅₀ (i.e. 50% binding concentration) of no more than: 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, or 0.07 nM by ELISA.

In certain embodiments, the bispecific antibody molecules provided herein specifically bind to human LAG-3 at an EC₅₀ (i.e. 50% binding concentration) of no more than: 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, or 0.2 nM by ELISA.

In certain embodiments, the bispecific antibody molecules provided herein specifically bind to cell surface human PD-1 at an EC₅₀ (i.e. 50% binding concentration) of no more than: 50 nM, 40 nM, 30 nM, 20 nM, 10 nM, 9 nM, 8 nM, 7 nM, 6 nM, 5 nM, 4 nM, 3 nM, 2 nM, or 1 nM by flow cytometry.

In certain embodiments, the bispecific antibody molecules provided herein specifically bind to cell surface human LAG-3 at an EC₅₀ (i.e. 50% binding concentration) of no more than: 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, or 2 nM by flow cytometry.

In certain embodiments, the bispecific antibody molecules provided herein cross-react with Cynomolgus monkey PD-1, for example, Cynomolgus monkey PD-1 expressed on a cell surface, or a soluble recombinant Cynomolgus monkey PD-1. In certain embodiments, the bispecific antibody molecules provided herein cross-react with Cynomolgus monkey LAG-3, for example, Cynomolgus monkey LAG-3 expressed on a cell surface, or a soluble recombinant Cynomolgus monkey LAG-3.

In certain embodiments, the bispecific antibody molecules provided herein specifically bind to cell surface Cynomolgus monkey PD-1 at an EC₅₀ of no more than: 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, or 0.3 nM by flow cytometry.

In certain embodiments, the bispecific antibody molecules provided herein specifically bind to cell surface Cynomolgus monkey LAG-3 with an EC₅₀ of no more than 21 nM, no more than: 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, or 6 nM by flow cytometry.

In some embodiments, the bispecific antibody molecules provided herein are capable of dual binding to human PD-1 and human LAG-3 with an EC₅₀ of no more than: 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, or 0.04 nM by ELISA.

In certain embodiments, the bispecific antibody molecules provided herein are capable of blocking the binding of PD-L1 to PD-1 at an IC₅₀ (i.e. 50% inhibiting concentration) of no more than: 20 nM, 18 nM, 16 nM, 14 nM, 12 nM, 10 nM, 9 nM, 8 nM, 7 nM, 6 nM, 5 nM, 4 nM, 3 nM, 2 nM, or 1 nM by flow cytometry.

In certain embodiments, the bispecific antibody molecules provided herein are capable of blocking the binding of LAG-3 to MEW II at an IC₅₀ of no more than: 50 nM, 40 nM, 30 nM, 20 nM, 10 nM, 9 nM, 8 nM, 7 nM, 6 nM, 5 nM, 4 nM, 3 nM, 2 nM, or 1 nM as determined by flow cytometry.

In certain embodiments, the bispecific antibody molecules provided herein do not cross-react with human CD4, CTLA-4 and CD28.

Binding affinity of the antigen-binding domains provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g. K_{D}) can be appropriately determined using suitable methods known in the art, including, for example, flow cytometry assay. In some embodiments, binding of the antibody to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, Calif.), wherein Bₘₐₓ refers to the maximum specific binding of the tested antibody to the antigen.

In some embodiments, the bispecific antibody molecules provided herein are capable of specifically binding to human PD-1 with a binding affinity (K_{D}) of no more than: 50×10⁻⁹ M, 40×10⁻⁹ M, 30×10⁻⁹ M, 20×10⁻⁹ M, 10×10⁻⁹ M, 9×10⁻⁹ M, 8×10⁻⁹ M, 7×10⁻⁹ M, 6×10⁻⁹ M, 5×10⁻⁹ M, 4×10⁻⁹ M, 3×10⁻⁹ M, or 2×10⁻⁹ M as measured by surface plasmon resonance (SPR).

In some embodiments, the bispecific antibody molecules provided herein are capable of specifically binding to human LAG-3 with a binding affinity (K_{D}) of no more than: 50×10⁻¹¹ M, 40×10⁻¹¹ M, 30×10⁻¹¹ M, 20×10⁻¹¹ M, 10×10⁻¹¹ M, 9×10⁻¹¹ M, 8×10⁻¹¹ M, 7×10⁻¹¹ M, 6×10⁻¹¹ M, 5×10⁻¹¹ M, 4×10⁻¹¹ M, 3×10⁻¹¹ M, or 2×10⁻¹¹ M as measured by surface plasmon resonance (SPR).

In certain embodiments, the bispecific antibody molecules provided herein block binding of human PD-1 to its ligand and thereby providing biological activity including, for example, inducing cytokine production from the activated T cells (such as CD4+ T cells and CD8+ T cells), inducing proliferation of activated T cells (such as CD4+ T cells and CD8+ T cells), and reversing T reg's suppressive function. Exemplary cytokines include IL-2 and IFNγ. The term “IL-2” refers to interleukin 2, a type of cytokine signaling molecule in the immune system that regulates the activities of white blood cells (e.g. leukocytes). The term “Interferon gamma (IFNγ)” is a cytokine that is produced by natural killer (NK), NK T cells, CD4+ and CD8+ T cells, which is a critical activator of macrophages and inducer of major histocompatibility complex (MHC) molecule expression. The cytokine production can be determined using methods known in the art, for example, by ELISA. Methods can also be used to detect proliferation of T cells, including [³H] thymidine incorporation assay.

In certain embodiments, the bispecific antibody molecules provided herein are capable of specifically enhancing nuclear factor of activated T-cells (NFAT) pathway in PD-1 expressing cells at an EC₅₀ of no more than: 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, or 0.4 nM as measured by reporter gene assay.

In certain embodiments, the bispecific antibody molecules provided herein are capable of specifically enhancing IL-2 pathway in LAG-3 expressing cells at an EC₅₀ of no more than: 20 nM, 18 nM, 16 nM, 14 nM, 12 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, or 0.3 nM, as measured by reporter gene assay.

In certain embodiments, the bispecific antibody molecules provided herein are capable of simultaneous stimulating cells from both the innate and the asdaptive immune system.

In certain embodiments, the bispecific antibody molecules provided herein block binding of human PD-1 to its ligand and thereby providing biological activity including, for example, inducing cytokine production from the activated T cells (such as CD4+ T cells and CD8+ T cells), inducing proliferation of activated T cells (such as CD4+ T cells and CD8+ T cells), and reversing Treg's suppressive function. Exemplary cytokines include IL-2 and IFNγ. The term “IL-2” refers to interleukin 2, a type of cytokine signaling molecule in the immune system that regulates the activities of white blood cells (e.g. leukocytes). The term “Interferon gamma (IFNγ)” is a cytokine that is produced by natural killer (NK), NK T cells, CD4+ and CD8+ T cells, which is a critical activator of macrophages and inducer of major histocompatibility complex (MHC) molecule expression. The cytokine production can be determined using methods known in the art, for example, by ELISA. Methods can also be used to detect proliferation of T cells, including [³H] thymidine incorporation assay.

C. Format of the Bispecific Antibody Molecule

Bispecific antibody fragments are antigen-binding fragments that are derived from an antibody but lack some or all of the antibody constant domains. Examples of such a bispecific antibody fragment include, for example, such as single domain antibody, Fv, Fab and diabody etc.

In certain embodiments, the bispecific antibody molecules as provided herein are based on the format of a “whole” antibody, such as whole IgG or IgG-like molecules

In certain embodiments, the bispecific antibody molecules as provided herein are in a bispecific format selected from IgG-appended antibodies with an additional antigen-binding moiety comprising IgG(H)-scFv; scFv-(H)IgG; IgG(L)-scFv; scFV-(L)IgG; IgG(L,H)-Fv; IgG(H)-V; V(H)-IgG; IgG(L)-V; V(L)-IgG; 2scFv-IgG; IgG-2scFv; scFv4-Ig; and scFv4-Ig. For detailed description of the bispecific antibody formats please see Spiess C., Zhai Q. and Carter P. J. (2015) Molecular Immunology 67: 95-106, which is incorporated herein by reference to its entirety.

The bispecific antibody molecules provided herein can be made with any suitable methods known in the art. In a conventional approach, two immunoglobulin heavy chain-light chain pairs having different antigen-binding specificities can be co-expressed in a host cell to produce bispecific antibodies in a recombinant way (see, for example, Milstein and Cuello, Nature, 305: 537 (1983)), followed by purification by affinity chromatography.

Recombinant approach may also be used, where sequences encoding the antibody heavy chain variable domains for the two specificities are respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g. Gruber et al., J. Immunol., 152:5368 (1994).)

D. Variants

The antigen-binding domains and bispecific antibody molecules provided herein also encompass various variants thereof. In certain embodiments, the variants comprise one or more modifications or substitutions in one or more CDR sequences of SEQ ID NOs: 1-6 and 11-16, as provided in Table 1 or Table 4, one or more variable region sequences (but not in any of the CDR sequences) of SEQ ID NOs: 17, 18, 7 and 8, as provided in Table 2 or Table 5, and/or the constant region (e.g. Fc region). Such variants retain specific binding affinity to LAG-3 and/or PD-1 of their parent antibodies, but have one or more desirable properties conferred by the modification(s) or substitution(s). For example, the antibody variants may have improved antigen-binding affinity, improved productivity, improved stability, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

In certain embodiments, the LAG-3-binding domains and/or the PD-1 binding domains provided herein comprise one or more amino acid residue substitutions in one or more CDR sequences, and/or one or more FR sequences, and/or one or more variable region sequences. In certain embodiments, a variant comprises no more than 10, 9, 8, 7, 6, 5, 4, 3, 2, or 1 substitutions in the CDR sequences and/or FR sequences and/or one or more variable region sequences in total.

In certain embodiments, the LAG-3-binding domains comprise 1, 2, or 3 CDR sequences having at least 80% (e.g. at least 85%, 88%, 90%, 91%, 92%, 93%, 94%, 95%, 96%, 97%, 98%, 99%) sequence identity to a sequence selected from SEQ ID NOs: 1-6, and in the meantime retain the binding affinity to LAG-3 at a level similar to or even higher than its parent antibody.

In certain embodiments, the anti-LAG-3-binding domains comprise one or more variable region sequences having at least 80% (e.g. at least 85%, 88%, 90%, 91%, 92%, 93%, 94%, 95%, 96%, 97%, 98%, 99%) sequence identity to SEQ ID NO: 7 or 8, and in the meantime retain the binding affinity to LAG-3 at a level similar to or even higher than its parent antibody. In some embodiments, a total of 1 to 10 amino acids have been substituted, inserted, or deleted in a variable region sequence of SEQ ID NO: 7 or 8. In some embodiments, the substitutions, insertions, or deletions occur in regions outside the CDRs (e.g., in the FRs).

In certain embodiments, the PD-1-binding domains comprise 1, 2, or 3 CDR sequences having at least 80% (e.g. at least 85%, 88%, 90%, 91%, 92%, 93%, 94%, 95%, 96%, 97%, 98%, 99%) sequence identity to a sequence selected from SEQ ID NOs: 11-16, and in the meantime retain the binding affinity to PD-1 at a level similar to or even higher than its parent antibody.

In certain embodiments, the PD-1-binding domains comprise one or more variable region sequences having at least 80% (e.g. at least 85%, 88%, 90%, 91%, 92%, 93%, 94%, 95%, 96%, 97%, 98%, 99%) sequence identity to SEQ ID NO: 17 or 18, and in the meantime retain the binding affinity to PD-1 at a level similar to or even higher than its parent antibody. In some embodiments, a total of 1 to 10 amino acids have been substituted, inserted, or deleted in a variable region sequence of SEQ ID NO: 17 or 18. In some embodiments, the substitutions, insertions, or deletions occur in regions outside the CDRs (e.g., in the FRs).

i. Glycosylation Variant

The antigen-binding domains and bispecific antibody molecules provided herein also encompass a glycosylation variant, which can be obtained to either increase or decrease the extent of glycosylation of the bispecific antibody molecules.

The antigen-binding domains and bispecific antibody molecules provided herein may comprise one or more amino acid residues with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

ii. Cysteine-Engineered Variant

The antigen-binding domains and bispecific antibody molecules also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibody polypeptides to introduce free cysteine residues are known in the art, see, for example, WO2006/034488.

iii. Fc Variant

The antigen-binding domains and bispecific antibody molecules provided herein also encompass an Fc variant, which comprises one or more amino acid residue modifications or substitutions at its Fc region and/or hinge region.

In certain embodiments, the antigen-binding domains and bispecific antibody molecules comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody molecule to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

In certain embodiments, the antigen-binding domains and bispecific antibody molecules comprise one or more amino acid substitution(s) that alters the antibody-dependent cellular cytotoxicity (ADCC). Certain amino acid residues at the Fc region (e.g. at the CH2 domain) can be substituted to provide for altered (e.g. enhanced, decreased, or depleted) ADCC activity. Alternatively or additionally, carbohydrate structures on the antibody can be changed to alter (e.g. enhance, decrease, or deplete) ADCC activity. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O, et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

In certain embodiments, the antigen-binding domains and bispecific antibody molecules comprise a human IgG4 constant region in which the 228^{th} amino acid residue is altered, for example from Ser228Pro (S228P, which may prevent or reduce strand exchange), and/or the 235^{th} amino acid residue is altered, for example from Leu235Glu (L235E, which may alter Fc receptor interactions.

In certain embodiments, the antigen-binding domains and bispecific antibody molecules comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fe region variants.

In certain embodiments, the antigen-binding domains and bispecific antibody molecules comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

E. Conjugates

In some embodiments, the bispecific antibody molecules further comprise a conjugate moiety. The conjugate moiety can be linked to the bispecific antibody molecules. A conjugate moiety is a non-proteinaceous moiety that can be attached to the bispecific antibody molecules. It is contemplated that a variety of conjugate moieties may be linked to the bispecific antibody molecules provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the bispecific antibody molecules by covalent binding, affinity binding, intercalation, coordinate binding, complexation, association, blending, or addition, among other methods.

In certain embodiments, the bispecific antibody molecules disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

In certain embodiments, the bispecific antibody molecules may be linked to a conjugate moiety indirectly, or through another conjugate moieties. For example, the bispecific antibody molecules may be conjugated to biotin, then indirectly conjugated to a second conjugate moiety that is conjugated to avidin. The conjugate moieties can be a clearance-modifying agent, a toxin (e.g., a chemotherapeutic agent), a detectable label (e.g., a radioactive isotope, a lanthanide, a luminescent label, a fluorescent label, or an enzyme-substrate label), or purification moiety.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the bispecific antibody molecule provided herein is used for a base for a conjugate.

F. Polynucleotides and Recombinant Methods

The present disclosure provides isolated polynucleotides that encode the bispecific antibody molecules provided herein.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In certain embodiments, the isolated polynucleotides comprise one or more nucleotide sequences as shown in SEQ ID NO: 9, 10, 19, 20, and/or a homologous sequence thereof having at least 80% (e.g. at least 85%, 88%, 90%, 92%, 93%, 94%, 95%, 96%, 97%, 98%, or 99%) sequence identity, and/or a variant thereof having only degenerate substitutions, and encodes the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the bispecific antibody molecule (e.g. including the sequences as shown in Table 3 and Table 6) can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors (e.g., expression vectors) containing the nucleic acid sequence provided herein encoding the bispecific antibody molecules, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the bispecific antibody molecule can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the vectors provided. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated bispecific antibody molecules provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is 293F cell.

Host cells are transformed with the above-described expression or cloning vectors for production of the bispecific antibody molecules and cultured in conventional nutrient media modified as appropriate for inducing promoters, selecting transformants, or amplifying the genes encoding the desired sequences. In another embodiment, the bispecific antibody molecules may be produced by homologous recombination known in the art.

The host cells used to produce the bispecific antibody molecule provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the bispecific antibody molecules can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the bispecific antibody molecules are secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The bispecific antibody molecules thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the bispecific antibody molecules. The suitability of protein A as an affinity ligand depends on the species and isotype of any immunoglobulin Fc domain that is present in the bispecific antibody molecules. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the bispecific antibody molecule comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody molecule of interest and contaminants may be subjected to low pH hydrophobic interaction chromatography using an elution buffer at a pH between about 2.5-4.5, preferably performed at low salt concentrations (e.g., from about 0-0.25M salt).

G. Pharmaceutical Composition

The present disclosure further provides pharmaceutical compositions comprising the bispecific antibody molecule and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising a bispecific antibody molecule and conjugates as provided herein decreases oxidation of the bispecific antibody molecule. This reduction in oxidation prevents or reduces loss of binding affinity, thereby improving antibody stability and maximizing shelf-life. Therefore, in certain embodiments compositions are provided that comprise one or more bispecific antibody molecules as disclosed herein and one or more antioxidants such as methionine. Further provided are methods for preventing oxidation of, extending the shelf-life of, and/or improving the efficacy of a bispecific antibody molecule as provided herein by mixing the bispecific antibody molecule with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving a bispecific antibody molecule as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the bispecific antibody molecule or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

H. Methods of Use

In another aspect, methods are provided to treat a condition in a subject that would benefit from up-regulation of immune response, comprising administering a therapeutically effective amount of the bispecific antibody molecule as provided herein to a subject in need thereof. The disease or condition that would benefit from up-regulation of an immune response is selected from the group consisting of cancer, a viral infection, a bacterial infection, a protozoan infection, a helminth infection, asthma associated with impaired airway tolerance, a neurological disease, multiple sclerosis, and an immunosuppressive disease.

Therapeutic methods are also provided, comprising: administering a therapeutically effective amount of the bispecific antibody molecule as provided herein to a subject in need thereof, thereby treating or preventing a PD-1 related and/or a LAG-3-related condition or a disorder.

PD-1-related conditions and disorders can be immune related disease or disorder, tumors and cancers, autoimmune diseases, or infectious disease. In certain embodiments, the PD-1-related conditions and disorders include tumors and cancers, for example, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma. In certain embodiments, the tumors and cancers are metastatic, especially metastatic tumors expressing PD-L1.

In certain embodiments, the PD-1-related conditions and disorders include autoimmune diseases. Autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune diabetes, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

In certain embodiments, the PD-1-related conditions and disorders include infectious disease. Infectious disease include, for example, chronic viral infection, for example, fungus infection, parasite/protozoan infection or chronic viral infection, for example, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV-1, HIV-2, cytomegalovirus, herpes simplex virus type I, herpes simplex virus type II, human papilloma virus, adenovirus, Kaposi West sarcoma associated herpes virus epidemics, thin ring virus (Torquetenovirus), human T lymphotrophic viruse I, human T lymphotrophic viruse II, varicella zoster, JC virus or BK virus.

In some embodiments, the subject has been identified as being likely to respond to a PD-1 antagonist. The presence or level of PD-L1 on an interested biological sample can be indicative of whether the subject from whom the biological sample is derived could likely respond to a PD-1 antagonist. Various methods can be used to determine the presence or level of PD-L1 in a test biological sample from the subject. For example, the test biological sample can be exposed to anti-PD-L1 antibody or antigen-binding fragment thereof, which binds to and detects the expressed PD-L1 protein. Alternatively, PD-L1 can also be detected at nucleic acid expression level, using methods such as quantitative Polymerase Chain Reaction (qPCR), reverse transcriptase PCR, microarray, Serial analysis of gene expression (SAGE), Fluorescence in situ hybridization (FISH), and the like. In some embodiments, the test sample is derived from a cancer cell or tissue, or tumor infiltrating immune cells. In certain embodiments, presence or upregulated level of the PD-L1 in the test biological sample indicates likelihood of responsiveness. The term “up-regulated” as used herein, refers to an overall increase of no less than 10%, 15%, 20%, 25%, 30%, 35%, 40%, 45%, 50%, 55%, 60%, 65%, 70%, 75%, 80% or greater, in the protein level of PD-L1 in the test sample, as compared to the PD-L1 protein level in a reference sample as detected using the same antibody. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor).

In some embodiments, the subject is resistant or has developed resistance to PD-1 antagonist therapy or PD-L1 inhibitor therapy. For example, the subject can be one who progressed (e.g., experienced tumor growth) during therapy with a PD-1 inhibitor (e.g., an antibody molecule as described herein) and/or a PD-L1 inhibitor (e.g., antibody molecule).

The present disclosure also provides therapeutic methods comprising: administering a therapeutically effective amount of the bispecific antibody molecule as provided herein to a subject in need thereof, thereby treating or preventing a LAG-3-related condition or a disorder. In some embodiment, the LAG-3-related condition or a disorder is cancer or infectious disease.

Examples of cancer include but are not limited to, lymphoma, bladder cancer, bone cancer, brain and central nervous system cancer, breast cancer, uterine or endometrial cancer, rectal cancer, esophageal cancer, head and neck cancer, anal cancer, gastrointestinal cancer, intra-epithelial neoplasm, kidney or renal cancer, leukemia, liver cancer, lung cancer (e.g. non-small cell lung cancer and small cell lung cancer), melanoma, myeloma, pancreatic cancer, prostate cancer, sarcoma, skin cancer, squamous cell cancer, stomach cancer, testicular cancer, vulval cancer, cancer of the endocrine system, cancer of the parathyroid gland, cancer of the adrenal gland, penile carcinoma, solid tumors of childhood, tumor angiogenesis, spinal axis tumor, pituitary adenoma, or epidermoid cancer.

Immune inhibitory molecules, e.g., PD-1 and LAG-3/TIM3, can regulate, e.g., synergistically regulate, T-cell function to promote tumoral immune escape. In certain embodiments, the bispecific molecule provided herein can be administered to treat a cancer, for example but not limited to, a solid tumor. In some embodiments, the subject is one who progressed (e.g., experienced tumor growth) during therapy with a PD-1 inhibitor (e.g., an antibody molecule as described herein) and/or a PD-L1 inhibitor (e.g., antibody molecule).

The therapeutically effective amount of an bispecific antibody molecule as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the bispecific antibody molecule as provided herein may be administered at a therapeutically effective dosage of about 0.01 mg/kg to about 100 mg/kg. In certain of these embodiments, the bispecific antibody molecule is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The bispecific antibody molecule disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the bispecific antibody molecules disclosed herein may be administered alone or in combination with one or more additional therapeutic means or agents. For example, the bispecific antibody molecules disclosed herein may be administered in combination with another therapeutic agent, for example, a chemotherapeutic agent or an anti-cancer drug.

In certain of these embodiments, an bispecific antibody molecule as disclosed herein that is administered in combination with one or more additional therapeutic agents may be administered simultaneously with the one or more additional therapeutic agents, and in certain of these embodiments the bispecific antibody molecule and the additional therapeutic agent(s) may be administered as part of the same pharmaceutical composition. However, a bispecific antibody molecule administered “in combination” with another therapeutic agent does not have to be administered simultaneously with or in the same composition as the agent. A bispecific antibody molecule administered prior to or after another agent is considered to be administered “in combination” with that agent as the phrase is used herein, even if the bispecific antibody molecule and second agent are administered via different routes. Where possible, additional therapeutic agents administered in combination with the bispecific antibody molecule disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of using the bispecific antibody molecule thereof.

In some embodiments, the present disclosure provides methods of detecting presence or amount of LAG-3 and/or PD-1 in a sample, comprising contacting the sample with the bispecific antibody molecule, and determining the presence or the amount of LAG-3 and/or PD-1 in the sample.

In some embodiments, the present disclosure also provides use of the bispecific antibody molecule provided herein in the manufacture of a medicament for treating a PD-1 and/or LAG-3 related disease or condition in a subject.

I. Advantages

The bispecific antibodies provided herein are advantageous over existing therapies in many aspects. For example, the bispecific antibodies provided herein can block both PD-1 and Lag-3 pathways, and they particularly inhibit Treg function and revive exhausted T cells. The bispecific antibodies provided herein are superior to monospecific anti-PD-1 antibodies, or monospecific anti-Lag-3 antibodies, or combination of monospecific anti-PD-1 antibodies and monospecific anti-Lag-3 antibodies. The bispecific antibodies provided herein are also advantageous in that they are cross-reactive to human, monkey PD-1 and Lag-3, but not murine PD-1. The bispecific antibodies provided herein also do not cross-react with human CTLA-4, CD28 or CD4 protein. The bispecific antibodies provided herein showed superior in vivo melanoma inhibition. Thus, the bispecific antibody may be used to treat the patients who are resistant to or relapse from anti-PD-1 therapy.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Generation and Characterization of Monoclonal Antibody of W3055-1.153.7

Fully human W3055-1.153.7 was obtained as described in PCT application No.: PCT/CN2016/094624, having a heavy chain variable region of SEQ ID NO: 17, a kappa light chain variable region of SEQ ID NO: 18, and a human IgG4 constant region. As disclosed in PCT application No.: PCT/CN2016/094624, the affinity of W3055-1.153.7 for recombinant human PD-1 was 2.79 nM by SPR. W3055-1.153.7 bound to cynomolgus monkey PD-1 but did not bind to murine PD-1 as measured by FACS. W3055-1.153.7 bound specifically to PD-1, but not to CD28 and CTLA4 of PD-1 family. The results of SPR assay and FACS for the binning test showed that the epitope on human PD-1 bound by W3055-1.153.7 was different from the existing PD-1 antibodies (i.e. benchmark antibody nivolumab (clone of 5C4 from BMS patent U.S. Pat. No. 9,084,776B2) and pembrolizumab (disclosed as clone hPD-1.09A in U.S. Pat. No. 8,354,509B2 and WO2008156712A1). [³H] thymidine incorporation assay showed that W3055-1.153.7 enhanced concentration dependent T cell proliferation.

Human CD4⁺ T Cells were stimulated with allogeneic dendritic cells (DCs) in the presence of W3055-1.153.7, which increased IL-2 secretion, IFNγ secretion in a dose manner by ELISA. W3055-1.153.7 enhanced concentration dependent CMV⁺- CD4⁺ T cell proliferation stimulated with CMV pp65 peptide-loaded autologous DC, as assessed by [³H]thymidine incorporation. W3055-1.153.7 abrogated Treg's suppressive function and restored responding T cell proliferation and IFNγ secretion, as assessed by [³H]thymidine incorporation.

W3055-1.153.7 has no ADCC and CDC function.

### Example 3: Generation and Characterization of Monoclonal Antibody of Human W3395-3.40.19 LAG-3 Ab

The monoclonal human LAG-3 antibody W3395-3.40.19 was generated as described in PCT/CN2019/076356. Generally, the OMT rats (transgenic rats having recombinant immunoglobulin loci, as described and produced in U.S. Pat. No. 8,907,157 B2) were immunized with human LAG-3 antigen to obtain antibodies in which both the framework and CDR regions are derived from human germline immunoglobulin sequences. The hybridoma generated by fusion of the immunized rat's lymph nodes and spleen with myeloma cell was isolated, selected and sub-cloned. The total RNA of the hybridoma was extracted and the cDNA was synthesized and amplified. VH and VL genes were re-amplified and cloned into expression vectors to create corresponding clones of the antibodies.

Binding affinity of W3395-3.40.19 to cell surface human LAG-3 has an EC50 value of 0.13 nM by FACS, which was much lower than that of BMK7 (0.61 nM, referred to as “H4sH15482P” in US 20170101472 A1) and BMK8 (0.90 nM, referred to as “BAP050-hum01” in WO2015138920 A1). In another affinity test, the binding affinity of W3395-3.40.19 to cell surface human LAG-3 has an KD value of 5.30E-11M by FACS, which was lower than that of BMK1 (2.70E-10M), BMK7 (5.80E-10M) and BMK8 (9.40E-10M).

W3395-3.40.19 blocked LAG-3 protein binding to MHC-II expressed on Raji cells with an EC₅₀ of 0.67 nM by FACS, which was superior over or comparable to that of BMK7 (EC₅₀ of 1.25 nM) and BMK8 (EC₅₀ of 0.88 nM). W3395-3.40.19 also blocked LAG-3 protein binding to LSECtin and Galectin-3. In an ELISA test, W3395-3.40.19 blocked LSECtin at an EC₅₀ of 0.51 nM, and blocked Galectin-3 at an EC₅₀ of 0.56 nM, which was superior over or comparable to BMK7 (EC₅₀ of 0.59 and 0.79 nM, respectively) and BMK8 (EC₅₀ of 1.06 and 1.07 nM, respectively). In the test of surface plasmon resonance (SPR), W3395-3.40.19 bound to human LAG-3 at a KD value of 1.06E-11M, which is lower than that of BMK1 (6.85E-10M), BMK7 (4.97E-10M), and BMK8 (7.97E-11M).

W3395-3.40.19 was tested to bind to cynomolgus LAG-3 at an EC₅₀ value of 3.92 nM, and weakly bound to murine LAG-3. W3395-3.40.19 did not bind to human CD4 protein.

In the epitope binning test, it was shown that W3395-3.40.19 has different epitope with all of BMK1, BMK7, and BMK5 (referred to as “BAP050-chi in WO2015138920 A1).

W3395-3.40.19 enhanced IL-2 pathway of Jurkat in reporter gene assay with an EC₅₀ of 0.21 nM, lower than that of BMK7 (2.65 nM) and BMK8 (65.3 nM). In the human allogeneic mixed lymphocyte reaction (MLR) test, W3395-3.40.19 enhanced IFN-γ secretion and T cell proliferation. W3395-3.40.19 did not mediate ADCC and CDC effects. Furthermore, W3395-3.40.19 was stable in fresh human serum for up to 14 days.

### Example 4. Construct and Characterization of Bispecific Antibodies

1. Antigen and Other Proteins Generation

1.1 Production of Antigens

Nucleic acids encoding human PD-1, human and mouse LAG-3 ECD (extracellular domain) were synthesized by Sangon Biotech. PD-1 or LAG-3 gene fragments were amplified from the synthesized nucleic acid and inserted into the expression vector pcDNA3.3 (ThermoFisher). The inserted PD-1 or LAG-3 gene fragment was further confirmed by DNA sequencing. Fusion proteins containing human LAG-3 ECD with various tags, including human Fc, mouse Fc, were obtained by transfection of human PD-1 or LAG-3 gene into 293F cells (ThermoFisher). The cells were cultured in FreeStyle 293 Expression Medium at 37° C., 5% CO₂. After 5 days of culture, supernatants were harvested from the culture of transiently transfected cells for protein purification. The fusion proteins were purified by protein A and/or SEC column. An untagged LAG-3 ECD protein was generated by cleavage of ECD-hFc fusion protein with a cut site using Factor Xa protease. Purified proteins were used for screening and characterization.

Mouse Fc-tagged human PD-L1 ECD, human CTLA-4 ECD and CD28 ECD were generated as above.

1.2 Production of Benchmark Antibodies

Gene sequences of anti-human PD-1 or LAG-3 benchmark antibodies (W339-BMK1 and W305-BMK1) were synthesized based on the information disclosed in patent applications US20110150892A1 (W339-BMK1 was referred to as “25F7”) and WO2006121168 (W305-BMK1 was referred to as “5C4”), respectively.

Sequences of anti-human PD-1×LAG-3 benchmark antibodies W365-BMK1, W365-BMK2 and W365-BMK3 were synthesized based on the information disclosed in patent applications WO2015200119A8 (W365-BMK1 was referred to as “SEQ25 & SEQ27”), WO2017087589A2 (W365-BMK2 was referred to as “SEQ110”) and WO2015200119A8 (W365-BMK3 was referred to as “SEQ 5 and 4”), respectively. The synthesized gene sequences were incorporated into plasmids pcDNA3.3. The cells transfected with the plasmids were cultured for 5 days and supernatant was collected for protein purification using Protein A column. The obtained benchmark antibodies were analyzed by SDS-PAGE and SEC, and then stored at −80° C.

2. Cell Line Generation

Human, cynomolgus PD-1 or LAG-3 transfectant cell lines were generated. Briefly, CHO-S or 293F cells were transfected with pcDNA3.3 expression vector containing full-length of human, cynomolgus PD-1 or LAG-3 using Lipofectamine transfection kit according to manufacturer's protocol, respectively. At 48-72 hours post transfection, the transfected cells were cultured in medium containing blasticidin for selection and tested for target expression. Human PD-1-expressing monoclonal cell line and cynomolgus LAG-3-expressing monoclonal cell line were obtained by limiting dilution.

Jurkat cell line was transfected with plasmids containing human full length PD-1/NFAT reporter or LAG-3/IL-2 reporter using Nucleofactor (Lonza). At 72 hours post transfection, the transfected cells were cultured in medium containing hygromycin for selection and tested for target expression. Jurkat cells expressing human PD-1 or LAG-3 along with stably integrated NFAT or IL-2 luciferase reporter gene were obtained after two months.

3. Bispecific Antibody Generation

1. Construct Expression Vectors

Construction of bispecific antibodies was conducted using molecular biology protocol. For W365-G14, briefly, DNA sequence encoding the light chain of anti-PD-1 antibody with scFv (VH-(G4S)3-VL) of anti-LAG3 antibody on the C-terminal, and the heavy chain of anti-PD1 antibody on the constant region of human IgG4 (S228P) heavy chain was cloned into modified pcDNA3.3 expression vector, respectively.

For W365-G15, DNA sequence encoding the light chain of anti-PD-1 antibody, and the heavy chain of anti-PD-1 antibody with scFv (VH-(G4S)3-VL) of anti-LAG3 antibody on the C-terminal of the constant region of human IgG4 (S228P) heavy chain was cloned into modified pcDNA3.3 expression vector, respectively.

4. In Vitro Characterization

4.1 Binding of W365-G14 and W365-G15 to Human PD-1 or LAG-3 Protein

Plates were coated with of W365-G14 and W365-G15 respectively overnight at 4° C. After blocking and washing, various concentrations of mouse Fc-tagged PD-1 protein or LAG-3 protein were added to the plates and incubated at room temperature for 1 hour. The plates were then washed and subsequently incubated with HRP-labeled goat anti-mouse IgG antibody for 1 hour. After washing, TMB substrate was added and the color reaction was stopped by 2M HCl. The absorbance at 450 nm was read using a microplate reader.

As shown in FIG. 1 and Table 9, the EC₅₀ of W365-G14 and W365-G15 for binding to PD-1 protein is comparable to the benchmarks.

TABLE 9
EC₅₀ of W365-G14 and W365-G15 bind to human PD-1 protein

| Antibody | EC₅₀ (nM) |
|---|---|
| W365-G14 | 0.07 |
| W365-G15 | 0.07 |
| W305-BMK1 | 0.09 |
| W365-BMK1 | 0.15 |
| W365-BMK2 | 0.18 |
| W365-BMK3 | 0.09 |

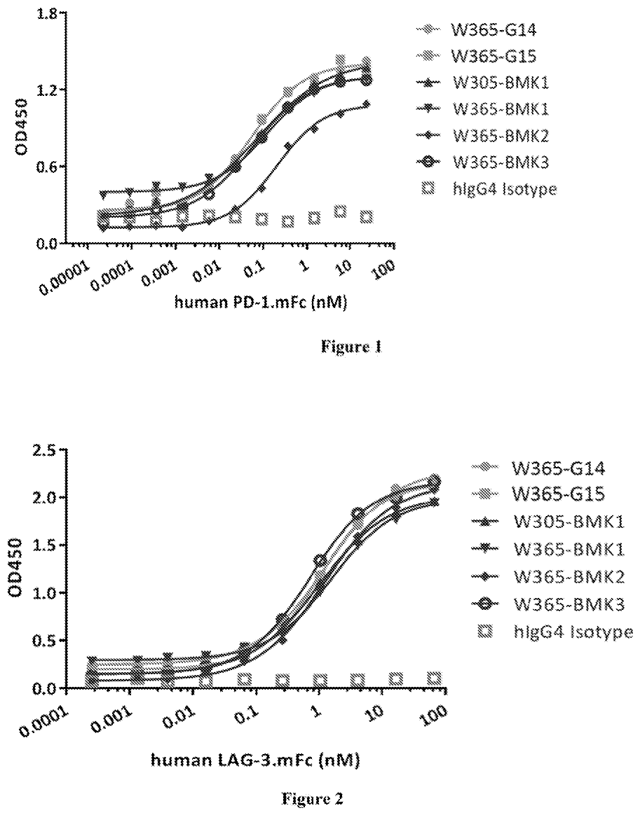

As shown in FIG. 2 and Table 10, the EC₅₀ of W365-G14 and W365-G15 for binding to LAG-3 protein is comparable to the benchmarks.

TABLE 10
EC₅₀ of W365-G14 and W365-G15 bind to human LAG-3 protein

| Antibody | EC₅₀ (nM) |
|---|---|
| W365-G14 | 0.27 |
| W365-G15 | 0.32 |
| W305-BMK1 | 0.23 |
| W365-BMK1 | 0.35 |
| W365-BMK2 | 0.28 |
| W365-BMK3 | 0.25 |

4.2 Binding of W365-G14 and W365-G15 to Cell Surface Human PD-1 or LAG-3

Human PD-1 expressing cells or transiently transfected human LAG-3 expressing 293F cells were incubated with various concentrations of W365-G14 and W365-G15, respectively. PE-labeled goat anti-human IgG antibody was used to detect the binding of W365-G14 and W365-G15 onto the cells. MFI of the cells was measured by flow cytometry and analyzed by FlowJo (version 7.6.1).

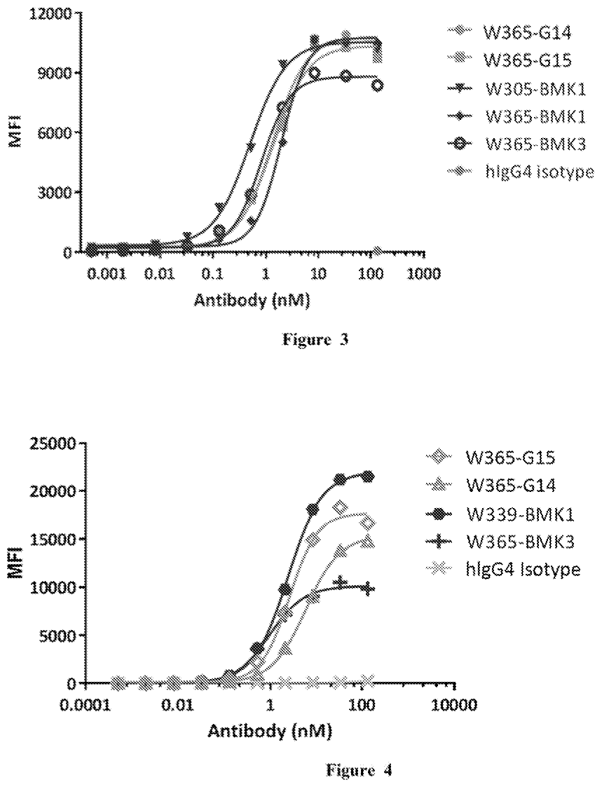

As shown in FIG. 3 and Table 11, the EC₅₀ of W365-G14 and W365-G15 for binding to cell surface human PD-1 is comparable to the benchmarks.

TABLE 11
EC₅₀ of W365-G14 and W365-G15 bind to cell surface human PD-1

| Antibody | EC₅₀ (nM) |
|---|---|
| W365-G14 | 1.32 |
| W365-G15 | 1.34 |
| W339-BMK1 | 0.50 |
| W365-BMK1 | 1.96 |
| W365-BMK2 | 0.82 |
| W365-BMK3 | 1.32 |

As shown in FIG. 4 and Table 12, the EC₅₀ of W365-G14 and W365-G15 for binding to cell surface human LAG-3 is comparable to the benchmarks.

TABLE 12
EC₅₀ of W365-G14 and W365-G15 bind to cell surface human LAG-3

| Antibody | EC₅₀ (nM) |
|---|---|
| W365-G14 | 2.57 |
| W365-G15 | 5.88 |
| W305-BMK1 | 2.40 |
| W365-BMK3 | 0.96 |

4.3 Binding of W365-G14 and W365-G15 to Cell Surface Cynomolgus PD-1 or LAG-3

Cynomolgus PD-1 or LAG-3 expressing 293F cells were incubated with various concentrations of W365-G14 and W365-G15, respectively. PE-labeled goat anti-human IgG antibody was used to detect the binding of W365-G14 and W365-G15 onto the cells. MFI of the cells was measured by flow cytometry and analyzed by FlowJo.

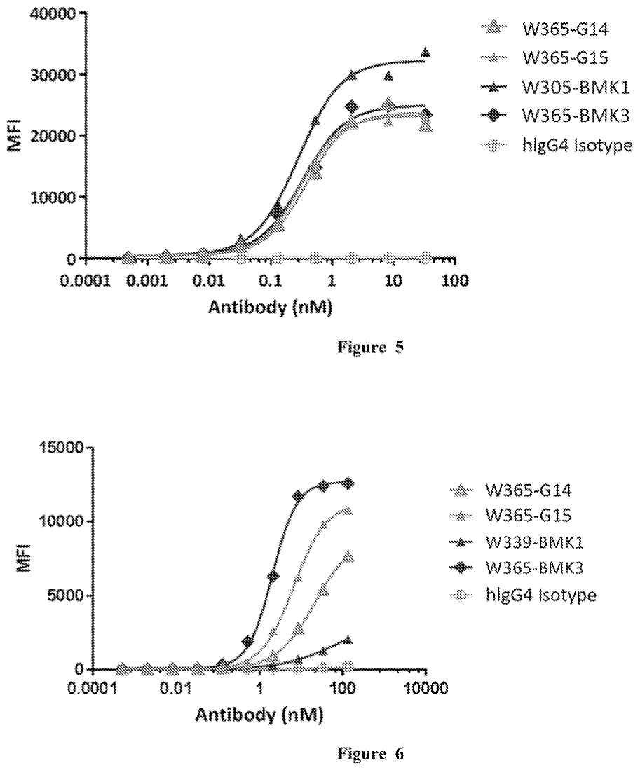

As shown in FIG. 5 and Table 13, the EC₅₀ of W365-G14 and W365-G15 for binding to cell surface cynomolgus PD-1 is comparable to the benchmarks.

TABLE 13
EC₅₀ of W365-G14 and W365-G15 bind to cell surface
cynomolgus PD-1

| Antibody | EC₅₀ (nM) |
|---|---|
| W365-G14 | 0.38 |
| W365-G15 | 0.31 |
| W305-BMK1 | 0.28 |
| W365-BMK3 | 0.33 |

As shown in FIG. 6 and Table 14, the EC₅₀ of W365-G14 and W365-G15 for binding to cell surface cynomolgus LAG-3 is better than W339-BMK1.

TABLE 14
EC₅₀ of W365-G14 and W365-G15 bind to cell surface
cynomolgus LAG-3

| Antibody | EC₅₀ (nM) |
|---|---|
| W365-G14 | 6.5 |
| W365-G15 | 20.9 |
| W339-BMK1 | Weak |
| W365-BMK3 | 2.0 |

4.4 Binding of W365-G14 and W365-G15 to Mouse PD-1 or LAG-3

For mouse PD-1 binding, plates were coated with W365-G14 and W365-G15 respectively overnight at 4° C. After blocking and washing, various concentrations of mouse PD-1 protein were added to the plates and incubated at room temperature for 1 hour. The plates were then washed and subsequently incubated with HRP-labeled goat anti-mouse IgG antibody for 1 hour. After washing, TMB substrate was added and the color reaction was stopped by 2M HCl. The absorbance at 450 nm was read using a microplate reader.

For mouse LAG-3 binding, plates were coated with mouse anti-His antibody overnight at 4° C. After blocking and washing, His-tagged LAG-3 protein was added to the wells. Various concentrations of W365-G14 and W365-G15 were added to the plates after wash and incubated at room temperature for 1 hour. The plates were then washed and subsequently incubated with HRP-labeled goat anti-human IgG antibody for 1 hour. After washing, TMB substrate was added and the color reaction was stopped by 2M HCl. The absorbance at 450 nm was read using a microplate reader.

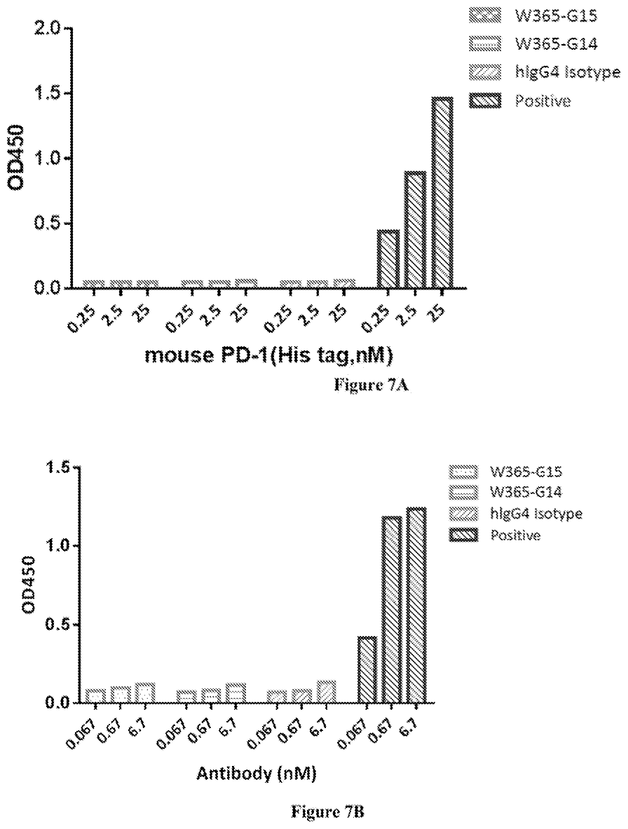

As shown in FIGS. 7A and 7B, W365-G14 and W365-G15 do not bind to mouse PD-1 or LAG-3.

4.5 Cross-Reactivity to Human CD4, CTLA-4 and CD28

Cross-reactivity to human CD4, CTLA-4 or CD28 was measured by ELISA. Plates were coated with human CD4, CTLA-4 or CD28 at 1 μg/mL overnight at 4° C. After blocking and washing, various concentrations of W365-G14 and W365-G15 were added to the plates and incubated at room temperature for 1 h. The plates were then washed and subsequently incubated with corresponding secondary antibody for 60 min. After washing, TMB substrate was added and the color reaction was stopped by 2M HCl.

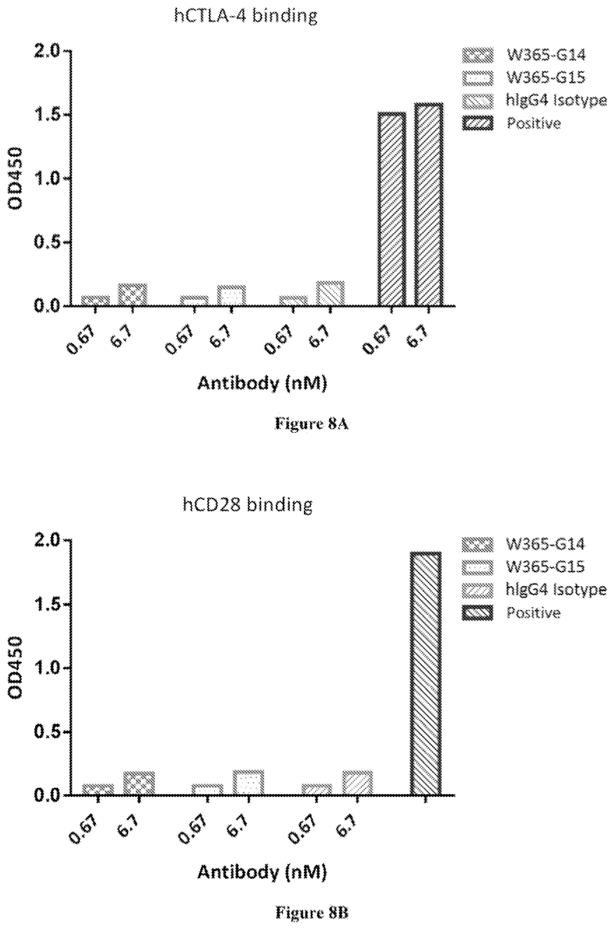

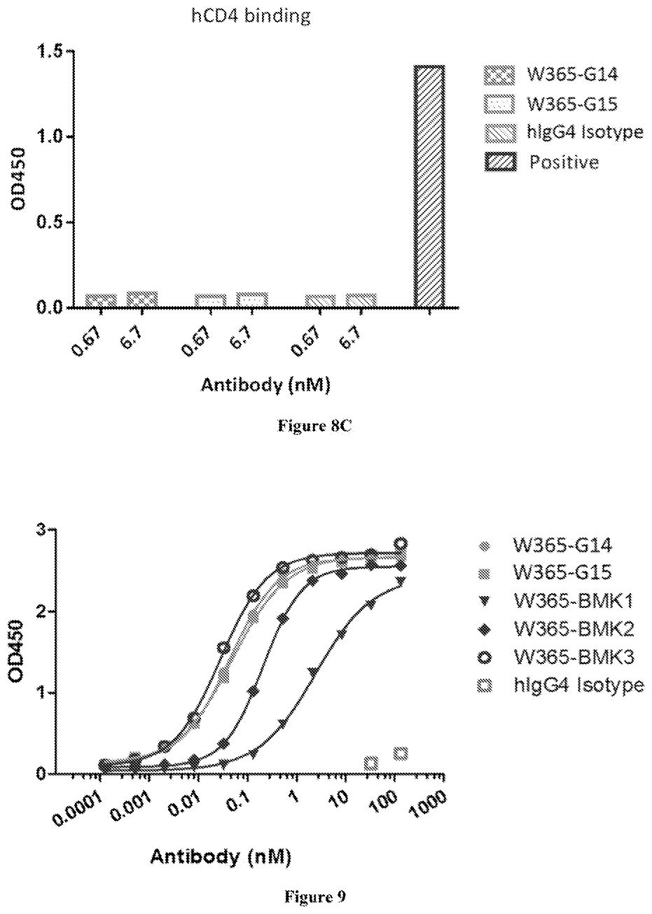

Results in FIGS. 8A, 8B and 8C indicate that W365-G14 and W365-G15 did not bind to human CTLA-4, CD28 or CD4 protein.

4.6 Affinity Test Against Human PD-1 and LAG-3 by SPR

Binding affinity of the bispecific antibodies to the antigen were determined by SPR assay using Biacore 8K. PD-1×LAG-3 antibodies were captured on an anti-human IgG Fc antibody immobilized CMS sensor chip (GE). His-tagged human PD-1 protein (MW: 40 KD) and cynomolgus PD-1 (MW: 40 KD) at different concentrations were injected over the sensor chip at a flow rate of 30 μL/min for an association phase of 120 s, followed by 800 s dissociation.

For affinity against human LAG-3, PD-1×LAG-3 antibodies were immobilized on a CMS sensor chip. Human LAG-3 without tag at different concentrations were injected over the sensor chip at a flow rate of 30 μL/min for an association phase of 180 s, followed by 3600 s dissociation using single-cycle kinetics method. The chip was regenerated with 10 mM glycine (pH 1.5).

The sensorgrams of blank surface and buffer channel were subtracted from the test sensorgrams. The experimental data was fitted by 1:1 model using Langmiur analysis. The results shown in Tables 15 and 16 indicated that both the affinity of W365-G14 and W365-G15 against human PD-1 and human LAG-3 were higher than the benchmarks.

TABLE 15
Affinity of W365-G14 and W365-G15 against human PD-1

| Ab | ka (1/Ms) | kd (1/s) | KD (M) |
|---|---|---|---|
| W365-G14 | 2.83E+05 | 6.95E−04 | 2.46E−09 |
| W365-G15 | 2.75E+05 | 5.92E−04 | 2.16E−09 |
| W305-BMK1 | 4.02E+05 | 1.35E−03 | 3.37E−09 |
| W365-BMK3 | 3.80E+05 | 1.36E−03 | 3.58E−09 |

TABLE 16
Affinity of W365-G14 and W365-G15 against human LAG-3

| Ab | ka (1/Ms) | kd (1/s) | KD (M) |
|---|---|---|---|
| W365-G14 | 3.83E+05 | <1.00E−05 | <2.61E−11 |
| W365-G15 | 7.14E+05 | 1.89E−05 | 2.65E−11 |
| W339-BMK1 | 4.87E+05 | 3.34E−04 | 6.85E−10 |
| W365-BMK3 | 1.02E+07 | 8.70E−04 | 8.51E−11 |

4.7 Dual Binding of W356-G14 and W365-G15 to Human PD-1 and LAG-3 Protein

Plates were coated with mouse Fc-tagged human PD-1 at 1 μg/mL overnight at 4° C. After blocking and washing, various concentrations of W365G-14 and W365-G15 were added to the plates and incubated at room temperature for 1 hour after washing. The plates were then washed and subsequently incubated with His-tagged LAG-3 protein for 1 hour. After washing, HRP-labeled anti-His antibody was added to the plate and incubated at room temperature for 1 hour. After washing, TMB substrate was added and the color reaction was stopped by 2M HCl. The absorbance at 450 nm was read using a microplate reader.

As shown in FIG. 9 and Table 17, the EC₅₀ of W365-G14 and W365-G15 for binding to LAG-3 protein is comparable to the W365-BMK3 and better than W365-BMK1 and BMK2.

TABLE 17
EC₅₀ of W365-G14 and W365-G15 bind to human PD-1
and LAG-3 protein

| Antibody | EC₅₀ (nM) |
|---|---|
| W365-G14 | 0.04 |
| W365-G15 | 0.05 |
| W365-BMK1 | 2.41 |
| W365-BMK2 | 0.20 |
| W365-BMK3 | 0.03 |

4.8 Blocking of PD-L1 Protein Binding to PD-1 Expressing Cells

Antibodies were serially diluted in 1% BSA-PBS and mixed with mouse Fc-tagged PD-L1 protein at 4° C. The mixture was transferred into the 96-well plates seeded with PD-1 expressing CHO-S cells. Goat anti-mouse IgG Fc-PE antibody was used to detect the binding of PD-L1 protein to PD-1 expressing cells. The MFI was evaluated by flow cytometry and analyzed by the software FlowJo.

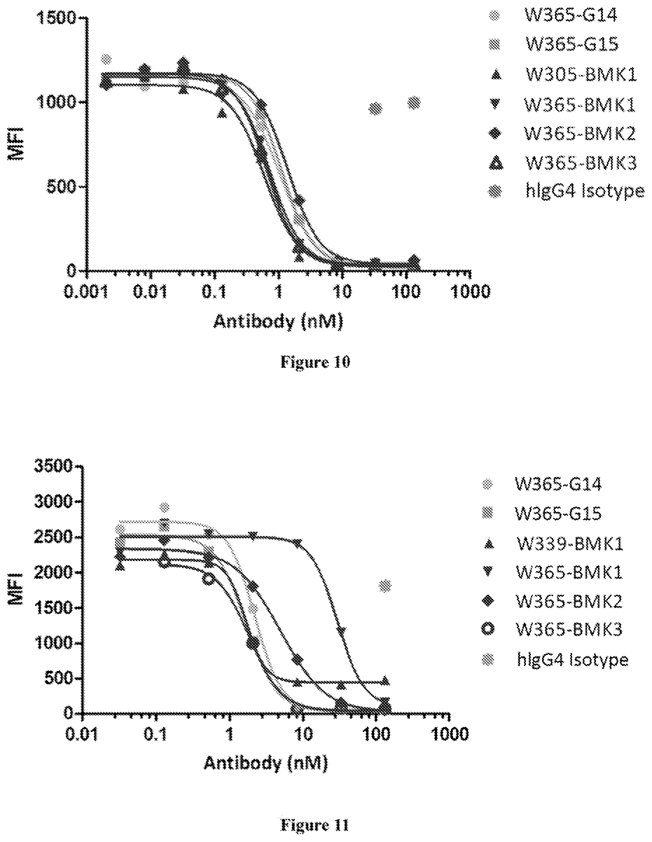

As shown in FIG. 10 and Table 18, the IC₅₀ of W365-G14 and W365-G15 for blocking the binding of PD-L1 to PD-1-expressed cells is comparable to the benchmarks.

TABLE 18
IC₅₀ of W365-G14 and W365-G15 block the binding of
PD-1 to PD-L1

| Antibody | IC₅₀ (nM) |
|---|---|
| W365-G14 | 1.01 |
| W365-G15 | 1.09 |
| W305-BMK1 | 0.59 |
| W365-BMK1 | 0.72 |
| W365-BMK2 | 1.36 |
| W365-BMK3 | 0.64 |

4.9 Blocking of LAG-3 Protein Binding to MHC-II Expressed on Raji Cells

Antibodies were serially diluted in 1% BSA-PBS and incubated with mouse Fc-tagged LAG-3 protein at 4° C. The mixture was transferred into the 96-well plates seeded with Raji cells which express MHC-II on the surface. Goat anti-mouse IgG Fc-PE antibody was used to detect the binding of LAG-3 protein to Raji cells. The MFI was evaluated by flow cytometry and analyzed by the software FlowJo.

As shown in FIG. 11 and Table 19, the IC₅₀ of W365 W365-G14 and W365-G15 for blocking the binding of LAG-3 to MHC-II-expressed Raji cells is better than W365-BMK1 and W365-BMK2 and comparable to other benchmarks.

TABLE 19
IC₅₀ of W365-G14 and W365-G15 block the binding of LAG-3
to MHC-II.

| Antibody | IC₅₀ (nM) |
|---|---|
| W365-G14 | 2.20 |
| W365-G15 | 1.68 |
| W339-BMK1 | 1.68 |
| W365-BMK1 | 30.0 |
| W365-BMK2 | 4.90 |
| W365-BMK3 | 1.88 |

4.10 Effects of W365-G14 and W365-G15 on PD-1 Expressing Jurkat with NFAT Reporter Gene

Jurkat cells expressing human PD-1 along with stably integrated NFAT luciferase reporter gene and human PD-L1 expressing artificial APC (antigen presenting cell) cells were seeded in 96-well plates. Testing antibodies were added to the cells. The plates were incubated for 6 hours at 37° C., 5% CO₂. After incubation, reconstituted luciferase substrate One-Glo was added and the luciferase intensity was measured by a microplate spectrophotometer.

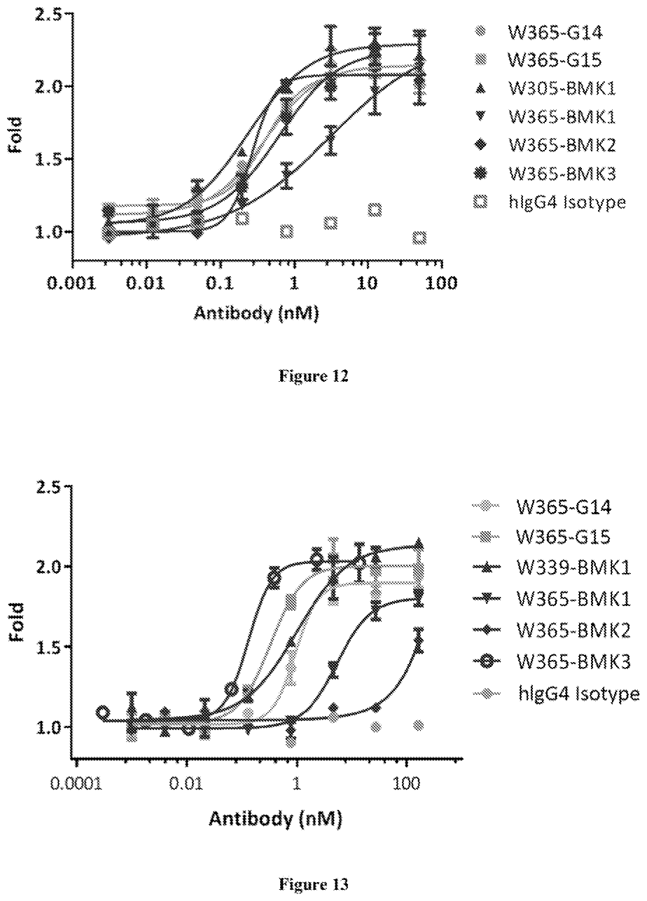

As demonstrated in FIG. 12, antibodies enhanced NFAT pathway of Jurkat in reporter gene assay. Further, as shown in Table 20, the EC₅₀ of W365-G14 and W365-G15 in this assay is better than W365-BMK1 and comparable to other benchmark antibodies.

TABLE 20
EC₅₀ of NFAT pathways enhancement in PD-1 expressing Jurkat

| Antibody | EC₅₀ (nM) |
|---|---|
| W365-G14 | 0.40 |
| W365-G15 | 0.41 |
| W305-BMK1 | 0.23 |
| W365-BMK1 | 3.10 |
| W365-BMK2 | 0.27 |
| W365-BMK3 | 0.62 |

4.11 Effects of W365-G14 and W365-G15 on LAG-3 Expressing Jurkat with IL-2 Reporter Gene

Jurkat cells expressing human LAG-3 along with stably integrated IL-2 luciferase reporter gene and Raji cells were seeded in 96-well plates in the presence of SEE (Staphylococcal enterotoxin E). Testing antibodies were added to the cells. The plates were incubated for overnight at 37° C., 5% CO₂. After incubation, reconstituted luciferase substrate One-Glo was added and the luciferase intensity was measured by a microplate spectrophotometer.

As demonstrated in FIG. 13 and Table 21, antibodies enhanced IL-2 pathway of Jurkat in reporter gene assay.

TABLE 21
EC₅₀ of IL-2 pathways enhancement in LAG-3 expressing Jurkat.

| Antibody | EC₅₀ (nM) |
|---|---|
| W365-G14 | 0.92 |
| W365-G15 | 0.32 |
| W339-BMK1 | 0.97 |
| W365-BMK1 | 5.48 |
| W365-BMK2 | Weak |
| W365-BMK3 | 0.13 |

4.12 Effects of W365-G15 on PD-1 and LAG-3 Expressing Jurkat with NFAT Reporter Gene

Full human LAG-3 plasmid was transiently transfected into Jurkat cells expressing human PD-1 along with stably integrated NFAT luciferase reporter gene. After 48 hours, the cells were seeded in 96-well plates along with Raji cells in the presence of SEE (Staphylococcal enterotoxin E). Testing antibodies were added to the cells. The plates were incubated for overnight at 37° C., 5% CO₂. After incubation, reconstituted luciferase substrate One-Glo was added and the luciferase intensity was measured by a microplate spectrophotometer.

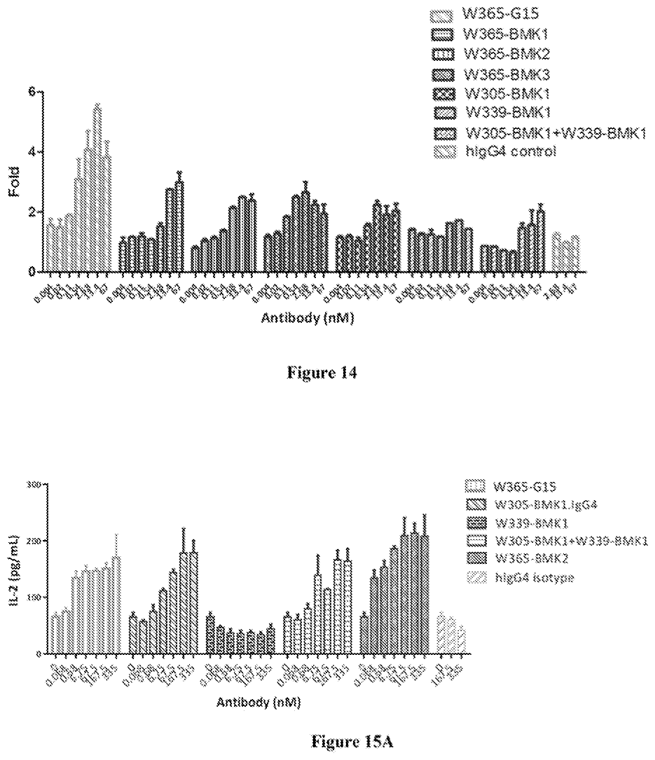

As demonstrated in FIG. 14, antibodies enhanced NFAT pathway of PD-1 and LAG-3 expressing Jurkat in reporter gene assay. The fold is higher than combination of W305-BMK1 and W339-BMK1 as well as other benchmark antibodies.

4.13 Effects of W365-G15 on Human Allogeneic Mixed Lymphocyte Reaction (MLR)

Human peripheral blood mononuclear cells (PBMCs) were freshly isolated from healthy donors using Ficoll-Paque PLUS gradient centrifugation. Monocytes were isolated using human monocyte enrichment kit according to the manufacturer's instructions. Cells were cultured in medium containing GM-CSF and IL-4 for 5 to 7 days to generate dendritic cells (DC). Human CD4⁺ T cells were isolated using human CD4⁺ T cell enrichment kit according to the manufacturer's protocol. Purified CD4⁺ T cells were co-cultured with allogeneic immature DCs (iDCs) in the presence of various concentrations of W365-G15 in 96-well plates. The plates were incubated at 37° C., 5% CO₂. Supernatants were harvested for IL-2 and IFN-γ test at day 3 and day 5, respectively. Human IL-2 and IFN-γ release were measured by ELISA using matched antibody pairs. Recombinant human IL-2 and IFN-γ were used as standards, respectively. The plates were pre-coated with capture antibody specific for human IL-2 or IFN-γ, respectively. After blocking, 50 μL of standards or samples were pipetted into each well and incubated for 2 hours at ambient temperature. Following removal of the unbound substances, the biotin-conjugated detecting antibody specific for corresponding cytokine was added to the wells and incubated for one hour. HRP-labeled streptavidin was then added to the wells for 30 minutes incubation at ambient temperature. The color was developed by dispensing 50 μL of TMB substrate, and then stopped by 50 of 2N HCl. The absorbance was read at 450 nM using a microplate spectrophotometer.

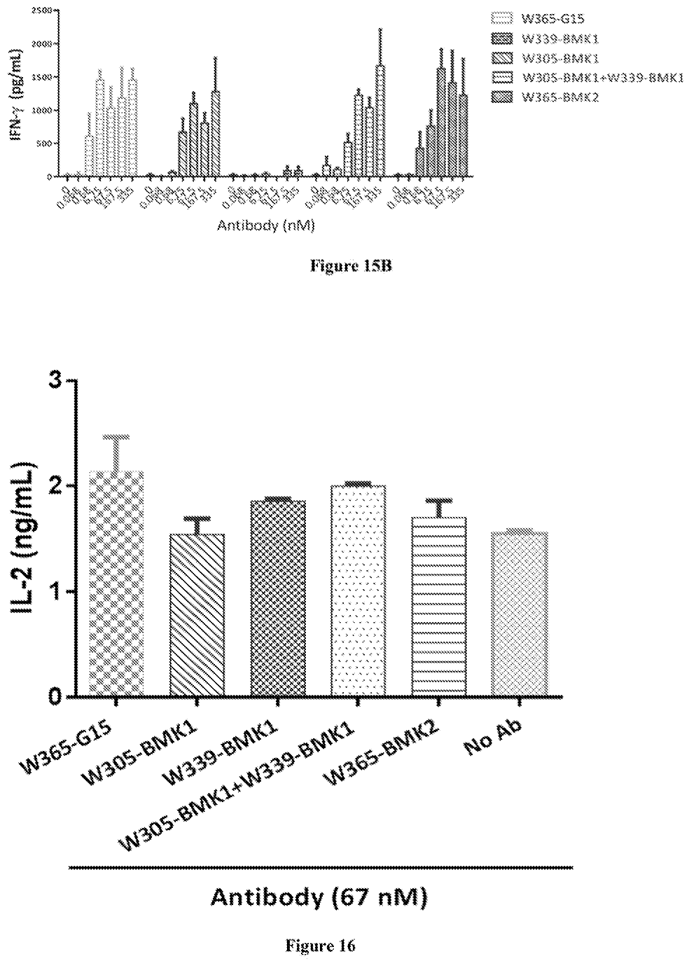

As demonstrated in FIGS. 15A and 15B, W365-G15 enhanced IL-2 and IFN-γ secretion in mixed lymphocyte reaction.

4.14 Effects of W365-G15 on Human PBMCs Activation

PBMCs and various concentrations of PD-1×LAG-3 antibodies were co-cultured in 96-well plates in the presence of -SEB. The plates were incubated at 37° C., 5% CO₂ for 3 days and supernatants were harvested for IL-2 test. Human IL-2 release was measured by ELISA. Human IL-2 release was measured by ELISA as described in section 4.13.

As demonstrated in FIG. 16, W365-G15 enhanced IL-2 and IFN-γ secretion in PBMCs stimulated with SEB.

4.15 Thermal Stability Test by Differential Scanning Fluorimetry (DSF)

Tm of antibodies was investigated using QuantStudio™ 7 Flex Real-Time PCR system (Applied Biosystems). 19 μL of antibody solution was mixed with 1 μL of 62.5×SYPRO Orange solution (Invitrogen) and transferred to a 96 well plate. The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tm. If a protein has multiple unfolding transitions, the first two Tm were reported, named as Tm1 and Tm2. Data collection and Tm calculation were conducted automatically by the operation software. Results are shown in Table 22.

TABLE 22
Tm of PD-1 × LAG-3 bispecific antibodies

| Antibody | PI | Buffer | Tₘ1 (° C.) | Tₘ2 (° C.) |
|---|---|---|---|---|
| W365-G15 | 6.37 | 20 mM Histidine, | 62.5 | 70.3 |
| | | 150 mM NaCl PH 6.0 | | |
| W365-G14 | 6.51 | 20 mM Histidine, | 60.0 | 69.1 |
| | | 150 mM NaCl PH 6.0 | | |

4.16 Serum Stability

The lead antibodies were incubated in freshly isolated human serum (serum content >95%) at 37° C. At indicated time points, aliquot of serum treated samples were removed from the incubator and snap frozen in liquid N₂, and then stored at −80° C. until ready for test. The samples were quickly thawed immediately prior to the stability test.

Plates were coated with mouse Fc-tagged human PD-1 at 1 μg/mL overnight at 4° C. After blocking and washing, various concentrations of W365-G14 and W365-G15 were added to the plates and incubated at room temperature for 1 hour after washing respectively. The plates were then washed and subsequently incubated with His-tagged LAG-3 protein for 1 hour. After washing, HRP-labeled mouse anti-His antibody was added to the plate and incubated at room temperature for 1 hour. After washing, TMB substrate was added and the color reaction was stopped by 2M HCl. The absorbance at 450 nm was read using a microplate reader.

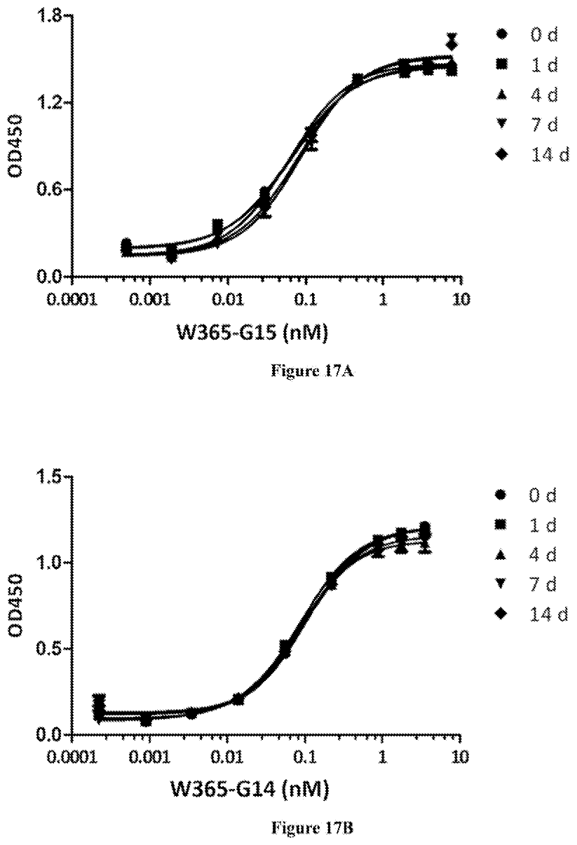

It is demonstrated in FIGS. 17A and 17B that W365-G14 and W365-G15 were stable in fresh human serum for up to 14 days.

5. In Vivo Characterization

In Vivo Anti-Tumor Activity of PD-1×LAG-3 Antibodies

Human PD-1/LAG-3 knock-in mouse (Biocytogen) and B16F10 tumor model were used to evaluate the ability of W365-G15 to inhibit the growth of tumor cells in vivo. Mouse were implanted subcutaneously with 1×10⁶ mouse melanoma cells B16F10 on day 0 and the mice were grouped (n=8) when the tumor reached 60-70 mm³.

On day 0, day 3, day 6 and day 9, the mice were intraperitoneally treated with PD-1 mAb (W305-BMK1) alone (10 mg/kg), LAG-3 mAb (W339-BMK1) alone (10 mg/kg), PD-1×LAG-3 antibody W365-G15 (13.1 mg/kg) or combination of W305-BMK1 (10 mg/kg) and W339-BMK1 (10 mg/kg). Human IgG4 isotype control antibody (10 mg/kg) was given as negative control.

Tumor volume and animal weight were measured for two weeks post injection. The tumor volume will be expressed in mm³ using the formula: V=0.5ab², where a and b are the long and short diameters of the tumor, respectively.

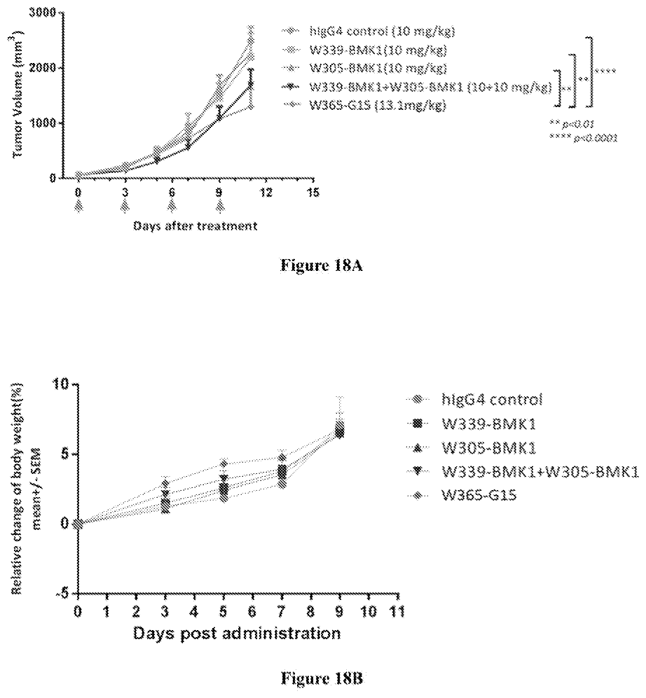

Tumor volume and survival curve of treated mice were shown in FIGS. 18A and 18B. The results show that the treatment with W339-BMK1 or W305-BMK1 antibody had little effect on B16F10 tumor growth inhibition in hLAG-3/hPD-1 knock-in mouse, while W365-G15 led to greater tumor growth inhibition than W339-BMK1 alone or W305-BMK1 alone. The efficacy of W365-G15 was comparable to combination of PD-1 and LAG-3 antibodies. Meanwhile, in FIG. 18B, the weight growth of each group indicated good safety without obvious toxicity.

For comparisons between the two groups, data were analyzed using T-test; for comparisons among three or more groups, data were analyzed using two-way ANOVA. Graphpad Prism was used for all data analysis. p<0.05 was considered as significant difference.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A bispecific antibody molecule comprising a LAG-3-binding domain and a PD-1-binding domain, wherein:
the LAG-3-binding domain comprises:
1, 2, or 3 heavy chain complementarity determining region (CDR) sequences selected from the group consisting of: SEQ ID NOs: 1-3; and/or
1, 2, or 3 light chain CDR sequences selected from the group consisting of: SEQ ID NOs: 4-6, and
the PD-1-binding domain comprises:
1, 2, or 3 heavy chain complementarity determining region (CDR) sequences selected from the group consisting of: SEQ ID NOs: 11-13; and/or
1, 2, or 3 light chain CDR sequences selected from the group consisting of: SEQ ID NOs: 14-16,
the LAG-3-binding domain comprises one independently selected from the group consisting of: a Fab and a single chain Fv antibody (scFv); and
the PD-1-binding domain comprises one independently selected from the group consisting of: a Fab and a scFv.

2-13. (canceled)

14. The bispecific antibody molecule of claim 1, wherein the LAG-3-binding domain further comprises one or more amino acid residue substitutions or modifications yet retains specific binding affinity to LAG-3, and/or the PD-1-binding domain further comprises one or more amino acid residue substitutions or modifications yet retains specific binding affinity to PD-1, wherein at least one of the substitutions or modifications is in one or more of the CDR sequences, and/or in one or more of the VH or VL sequences but not in any of the CDR sequences.

15. (canceled)

16. The bispecific antibody molecule of claim 1, wherein the bispecific antibody molecule further comprises an immunoglobulin (Ig) constant region, optionally a constant region of human IgG, or optionally a constant region of human IgG4.

17-18. (canceled)

19. The bispecific antibody molecule of claim 1, wherein the LAG-3-binding scFv comprises the sequence of SEQ ID NO: 38, and the PD-1-binding Fab comprises a heavy chain variable region comprising the sequence of SEQ ID NO: 17 and a light chain variable region comprising the sequence of SEQ ID NO: 18.

20-26. (canceled)

27. The bispecific antibody molecule of claim 1 linked to one or more conjugate moieties.

28. (canceled)

29. A pharmaceutical composition comprising the bispecific antibody molecule of claim 1, and a pharmaceutically acceptable carrier.

30. An isolated polynucleotide comprising a nucleic acid sequence encoding the heavy chain and/or light chain of the bispecific antibody molecule of claim 1.

31. (canceled)

32. A vector comprising the isolated polynucleotide of claim 30.

33. A host cell comprising the vector of claim 32.

34. A method of producing the bispecific antibody molecule of claim 1, comprising culturing a host cell having a vector under the condition at which the vector is expressed, the vector comprises a polynucleotide encoding the bispecific antibody molecule.

35. A method of treating a disease or condition in a subject, comprising administering to the subject a therapeutically effective amount of the bispecific antibody molecule of claim 1, wherein the disease or condition is characterized by at least one of the following: PD-1-related, LAG-3-related, and would benefit from upregulation of an immune response.

36. The method of claim 35, wherein the disease or condition is selected from cancer, infectious disease including a viral infection, a bacterial infection, a protozoan infection, a helminth infection, asthma associated with impaired airway tolerance, a neurological disease, multiple sclerosis, and an immunosuppressive disease.

37-45. (canceled)

46. The bispecific antibody molecule of claim 1, wherein the LAG-3-binding domain comprises a scFv and the PD-1-binding domain comprises a Fab, and the scFv is operably linked to:
(a) the C terminus of the heavy chain of the Fab, or
(b) the C terminus of the light chain of the Fab.

47. The bispecific antibody molecule of claim 1, wherein:
(a) the LAG-3-binding domain comprises at least one of the following variable regions:
a heavy chain variable region, which comprises SEQ ID NO: 7 or a homologous sequence thereof having at least 80% sequence identity yet retaining specific binding affinity to LAG-3; and
a light chain variable region, which comprises SEQ ID NO: 8 or a homologous sequence thereof having at least 80% sequence identity yet retaining specific binding affinity to LAG-3;
and/or
(b) the PD-1-binding domain comprises at least one of the following variable regions:
a heavy chain variable region, which comprises SEQ ID NO: 17 or a homologous sequence thereof having at least 80% sequence identity yet retaining specific binding affinity to PD-1; and
a light chain variable region, which comprises SEQ ID NO: 18 or a homologous sequence thereof having at least 80% sequence identity yet retaining specific binding affinity to PD-1.

48. The bispecific antibody molecule of claim 1, wherein the bispecific antibody comprises:
(a) a heavy chain in the format of VH(anti-PD-1)-CH1-Hinge-CH2-CH3-spacer-scFv(anti-LAG-3), which is associated with the light chain VL(anti-PD-1)-CL; or
(b) a heavy chain in the format of VH(anti-PD-1)-CH1-Hinge-CH2-CH3, which is associated with the light chain in the format of VL(anti-PD-1)-CL-spacer-scFv(anti-LAG-3).

49. The bispecific antibody molecule of claim 1, wherein the bispecific antibody molecule has at least one of the following properties:
(a) capable of binding to human PD-1, human LAG-3, cynomolgus PD-1 and cynomolgus LAG-3;
(b) do not bind to mouse PD-1 or LAG-3;
(c) have no cross reactivity to human CTLA-4, CD28 or CD4 protein;
(d) capable of dual binding to human PD-1 and LAG-3 protein;
(e) enhance IL-2 pathway of Jurkat in reporter gene assay;
(f) enhance NFAT pathway of PD-1 and LAG-3 expressing Jurkat in reporter gene assay; and
(g) significantly inhibit tumor growth in vivo.

50. The bispecific antibody molecule of claim 1, wherein the bispecific antibody molecule has an KD value of no more than 3×10-9 M for human PD-1, and an KD value of no more than 5×10-11 M for human LAG-3, as measured by SPR.

51. The bispecific antibody molecule of claim 1, comprising:
(a) a heavy chain comprising the sequence of SEQ ID NO: 33 and a light chain comprising the sequence of SEQ ID NO: 34; or
(b) a heavy chain comprising the sequence of SEQ ID NO: 31 and a light chain comprising the sequence of SEQ ID NO: 32.

52. The method of claim 36, wherein the cancer is melanoma, lymphoma, lung cancer, liver cancer, cervical cancer, colon cancer, breast cancer, ovarian cancer, pancreatic cancer, glioblastoma, prostate cancer, esophageal cancer or gastric cancer.

53. A method of modulating LAG-3 activity in a LAG-3-expressing cell, comprising exposing the LAG-3-expressing cell to the bispecific antibody molecule of claim 1.
